(19) United States
(12) Patent Application Publication
(10) Pub. No.: US 2024/0000342 A1
(43) Pub. Date: Jan. 4, 2024

(54) METHOD FOR DECREASING MELTDOWN INCIDENCE AND SEVERITY IN NEURODEVELOPMENTAL DISORDERS

(71) Applicant: Montera d/b/a Forta, San Francisco, CA (US)

(72) Inventors: Madalina CIOBANU, San Francisco, CA (US); Rahul THAPA, San Francisco, CA (US); Anurag GARIKIPATI, San Francisco, CA (US); Jenish MAHARJAN, San Francisco, CA (US); Navan Preet SINGH, San Francisco, CA (US); Shobhan THAKKAR, San Francisco, CA (US)

(21) Appl. No.: 18/216,358

(22) Filed: Jun. 29, 2023

Related U.S. Application Data
(60) Provisional application No. 63/358,000, filed on Jul. 1, 2022.

(51) Int. Cl. A61B 5/11; G06F 3/01; G06F 3/16

(52) U.S. Cl. CPC A61B 5/11; G06F 3/011; G06F 3/167

(57) ABSTRACT

A method comprising (a) acquiring sensor data from wearable sensors worn by a subject, wherein the sensor data comprise motion, sound, and/or physiological data; (b) comparing the sensor data with target data; (c) determining: that the motion data of the subject is equal to or exceed the target motion data; that the sound data of the subject is equal to or exceed the target sound data; and/or that the physiological data of the subject is equal to or exceed the target physiological data; and (d) responsive to step (c), delivering audible sound therapy to the subject, wherein the audible sound therapy comprises a familiar audio sound track which is repeated at least until it is determined: that the target motion data exceed the motion data; that the target sound data exceed the sound data; and/or that the target physiological data exceed the physiological data. Steps (b)-(c) can encompass machine learning.

### CROSS-REFERENCE TO RELATED APPLICATIONS

This application claims priority to U.S. Provisional Application Ser. No. 63/358,000 filed on Jul. 1, 2022 and entitled “Method for Decreasing Meltdown Incidence and Severity in Neurodevelopmental Disorders” by Madalina Ciobanu, et al., which is incorporated herein by reference in its entirety.

### TECHNICAL FIELD

The present disclosure relates to methods of improving the quality of life for individuals with autism spectrum disorder (ASD). More specifically, it relates to methods of decreasing meltdown incidence and/or severity in individuals having ASD.

### BACKGROUND

Autism spectrum disorder (ASD) is a complex neurodevelopmental disorder which expresses heterogeneously in afflicted individuals, although a couple of essential features are always present: social communication impairment as well as restricted interests and repetitive behaviors. It is estimated that currently about 1 in 100 children worldwide are diagnosed with ASD, while the Centers for Disease Control and Prevention (CDC) estimates based on 2018 data that about 1 in 44 8-year-old children have been identified with ASD in the United States. ASD occurs across all geographic regions and socio-economic groups, wherein the discrepancies in officially diagnosed ASD from one demographic to another can largely be attributed to the difficulty of the diagnostic process as well as lack of awareness, rather than an actual difference in disorder prevalence between demographics.

Individuals with ASD can display episodic meltdowns, generally triggered by a variety of factors such as stress, social demands, frustration, embarrassment, challenges with communication, emotional triggers, overwhelming aversive sensory stimuli, etc. ASD meltdowns are preceded by a pre-meltdown stage, where the individual experiences an increase in agitation, which is also referred to as the rumbling stage. There are several types of interventions that could be implemented in the rumbling stage, wherein such interventions could diminish the meltdowns, both in frequency and severity. For example, removing an individual from a stressful environment, redirecting the individual towards a routine type of activity, moving the individual to a place with a specific structure, etc. can reduce the meltdown intensity or eliminate it altogether. However, these conventional interventions require a caretaker to perform, and are effective only if the caretaker recognizes the rumbling stage. The rumbling stage can be characterized by behaviors which can vary greatly from an individual to another, and at times may appear to be minor such as nail biting, tensing muscles, or otherwise indicating discomfort; all of which can be inadvertently missed by a caretaker. Thus, there is an ongoing need to develop methods for recognizing pre-meltdown behaviors, as well as methods of intervening in pre-meltdown behaviors in order to decrease meltdown incidence and/or severity.

### BRIEF SUMMARY

Disclosed herein is a method comprising (a) acquiring sensor data from one or more wearable sensors configured to be worn by a subject, wherein the sensor data comprise motion data, sound data, physiological data, or combinations thereof of the subject; (b) comparing the sensor data of the subject with target data, wherein the motion data, the sound data, the physiological data, or combinations thereof of the subject are compared with target motion data, target sound data, target physiological data, or combinations thereof, respectively; (c) determining, in any sequence, at least one of the following: that the motion data of the subject is equal to or exceed the target motion data; that the sound data of the subject is equal to or exceed the target sound data; and that the physiological data of the subject is equal to or exceed the target physiological data; and (d) responsive to step (c), delivering audible sound therapy to the subject, wherein the audible sound therapy comprises an audio sound track, wherein the audio sound track is characterized by a track rhythm and by a track beat, wherein the audio sound track is familiar to the subject, and wherein the audio sound track is repeated at least until it is determined, in any sequence, at least one of the following: that the target motion data exceed the motion data of the subject; that the target sound data exceed the sound data of the subject; and that the target physiological data exceed the physiological data of the subject.

Further disclosed herein is a method comprising (a) acquiring sensor data from one or more wearable sensors configured to be worn by a subject, wherein the subject has autism spectrum disorder (ASD), and wherein the sensor data are time-series data; (b) transmitting the sensor data from the one or more wearable sensors to at least one computing device; (c) filtering, by the at least one computing device, the sensor data to yield filtered sensor data; (d) processing, by the at least one computing device, the filtered sensor data to yield processed sensor data, wherein the processed sensor data are tabular data; (e) evaluating the sensor data of the subject with respect to target data; wherein evaluating the sensor data of the subject with respect to the target data comprises providing, by the at least one computing device, an input to an algorithm based on at least one machine learning model that is trained with sensor data, filtered sensor data, processed sensor data, or combinations thereof from the subject and/or with data from at least one additional subject, wherein the algorithm is configured to evaluate the sensor data of the subject with respect to the target data, provide an evaluation score (e.g., prediction score), and compare the evaluation score with a threshold score value; (f) determining, by the at least one computing device, that the evaluation score is equal to or exceeds a threshold score value, wherein the evaluation score being equal to or exceeding a threshold score value corresponds to the sensor data of the subject being equal to or exceeding the target data, and wherein the sensor data being equal to or exceeding target data correlates with the onset of a pre-meltdown stage for the subject; and (g) responsive to step (f), delivering, by the at least one computing device, audible sound therapy to the subject, wherein the audible sound therapy comprises an audio sound track, wherein the audio sound track is characterized by a track rhythm and by a track beat, wherein the audio sound track is familiar to the subject, and wherein the audio sound track is repeated at least until it is determined, by the at least one computing device, that the threshold score value is equal to or exceeds an evaluation score; and wherein delivering the audible sound therapy to the subject prevents the onset of a meltdown stage for the subject or decreases the severity of a meltdown stage for the subject.

### BRIEF DESCRIPTION OF THE DRAWINGS

For a detailed description of the preferred aspects of the disclosed processes and systems, reference will now be made to the accompanying drawings in which:

FIG. 1 displays a flow diagram of a process for decreasing meltdown incidence and/or severity in a subject having a neurodevelopmental disorder;

FIG. 2 displays another flow diagram of a process for decreasing meltdown incidence and/or severity in a subject having a neurodevelopmental disorder;

FIG. 3 displays a diagram of system architecture for a process for decreasing meltdown incidence and/or severity in a subject having a neurodevelopmental disorder;

FIG. 4 displays another diagram of system architecture for a process for decreasing meltdown incidence and/or severity in a subject having a neurodevelopmental disorder;

FIG. 5 displays a diagram of a machine learning prediction model;

FIG. 6 displays a diagram of training a machine learning model;

FIG. 7 is a schematic representation of a computing system by way of which a machine-learning model may be employed; and

FIG. 8 is a schematic representation of a machine-learning model.

### DETAILED DESCRIPTION

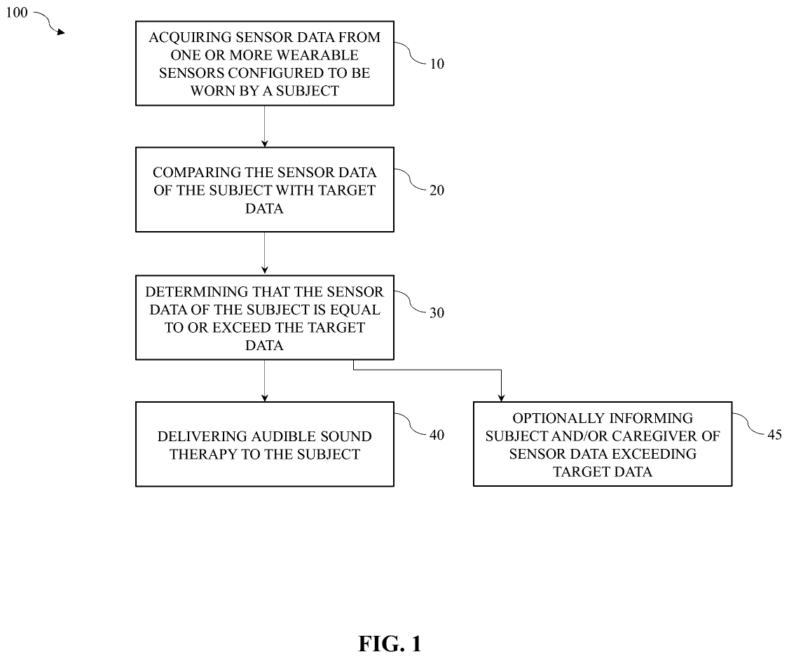

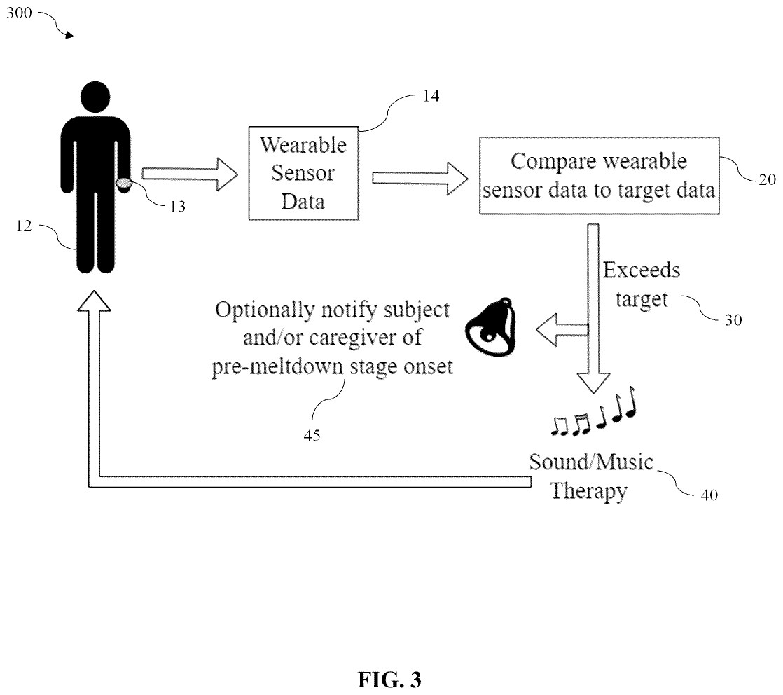

Disclosed herein, and with reference to FIGS. 1 and 3, is a method 100 for decreasing meltdown incidence and/or severity in a subject having a neurodevelopmental disorder, the method comprising: a step 10 of acquiring sensor data 14 from one or more wearable sensors 13 configured to be worn by a subject 12, wherein the sensor data comprise motion data, sound data, physiological data, or combinations thereof of the subject; a step 20 of comparing the sensor data of the subject with target data; a step 30 determining that at least one of the sensor data is equal to or exceed a corresponding target data; and a step 40, which is responsive to step 30, of delivering audible sound therapy to the subject, wherein the audible sound therapy comprises a familiar audio sound track, and wherein the audio sound track is repeated at least until it is determined that the target data exceed the corresponding sensor data of the subject. For purposes of the disclosure herein, the term “target data” refers to a level of sensor data that triggers the implementation of a method that for decreasing meltdown incidence and/or severity in a subject having a neurodevelopmental disorder, as disclosed herein. For purposes of the disclosure herein, the term “exceed” with respect to the relationship between sensor data and target data refers to the sensor data indicating that a particular type of sensor data approaches a concern level, wherein this concern level may be a greater value than the value for a non-concerning level for certain type of sensor data, while for other type of data the concern value may have a lower value than for a non-concerning level. For example, heart rate data may have a concern level value that is greater than a usual heart rate value for the subject, and in this particular case, when the concerning heart rate data exceeds the target heart rate, the concerning heart rate data is greater in value than the target heart rate. As another example, for an individual that is habitually stimming with a high frequency motion, motion sensor data (e.g., motion frequency data) may have a concern level that is lower than a usual motion frequency for the subject, and in this particular case, when the concerning motion frequency data exceeds the target motion frequency, the concerning motion frequency is lower in value than the target motion frequency. In an aspect, the audible sound therapy may end when the sensor data returns to usual or habitual values under the target data values. Alternatively, the audible sound therapy may continue once the sensor data returns to usual or habitual values under the target data values. In some aspects, the method 100 can further comprise a step 45 of optionally informing the subject and/or a caregiver of the sensor data exceeding the target data (e.g., informing of the onset of a pre-meltdown stage). The caregiver can be a trusted adult, a parent, a relative, a clinician, a physician, a healthcare professional, a therapist, etc. The audible sound therapy may be started by the subject subsequent to the subject being informed of the onset of a pre-meltdown stage. The audible sound therapy may be paused or interrupted by the subject and/or a caregiver, as necessary or desired. In other words, the subject and/or a caregiver can prevent the therapy from starting or can turn off the therapy, as necessary or desired. In some aspects, the subject may start the audible sound therapy without being informed of the onset of a pre-meltdown stage. In an aspect, the audible sound therapy may have a predetermined volume (e.g., loud, soft, etc.) and volume profile: the volume may either stay substantially constant or change as necessary or desired while the audible sound therapy is delivered. The volume and volume profile during the audible sound therapy may be pre-determined (preset) or may be changed by the subject as necessary or desired during the audible sound therapy. In some aspects, the volume and volume profile during the audible sound therapy may be changed by the caregiver as necessary or desired during the audible sound therapy. In an aspect, the neurodevelopmental disorder can comprise autism spectrum disorder (ASD), sensory processing disorder (SPD), or both ASD and SPD. In an aspect, the neurodevelopmental disorder can comprise any suitable disorder wherein the subject can exhibit meltdowns as disclosed herein. While the current disclosure will be discussed in detail in the context of the neurodevelopmental disorder comprising ASD, it should be understood that the methods and systems disclosed herein can be used for delivering therapy to a subject having any suitable disorder where the subject can exhibit meltdowns as disclosed herein, for example SPD, alternatively bipolar disorder (for example during manic episodes), alternatively panic disorders, alternatively anxiety disorders, alternatively obsessive disorders (e.g., obsessive-compulsive disorder (OCD)), alternatively paranoia, or alternatively personality disorders (e.g., borderline personality disorder, dissociative identity disorder).

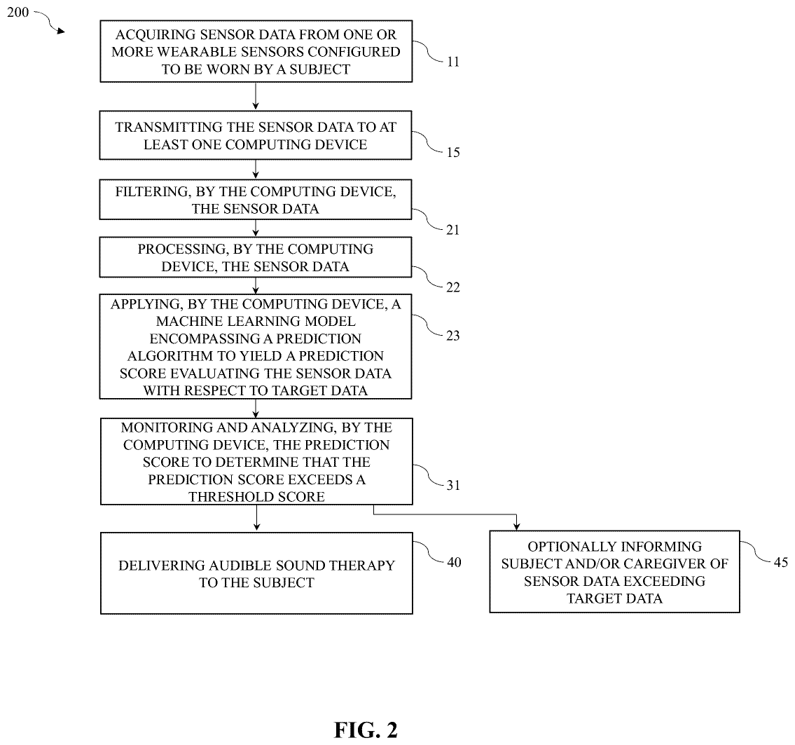

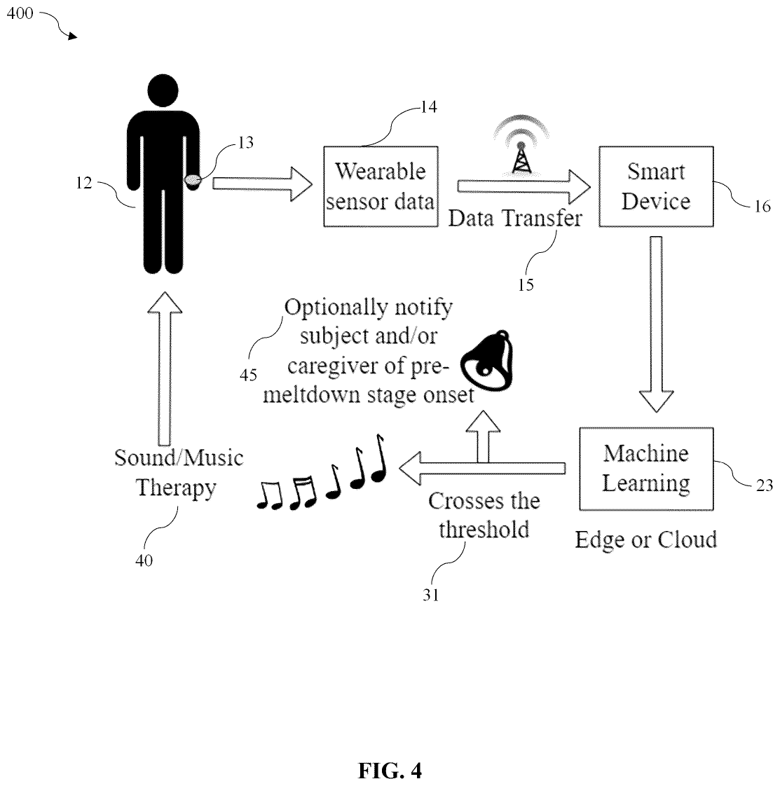

Further disclosed herein, and with reference to FIGS. 2 and 4, is a method 200 for decreasing meltdown incidence and/or severity in a subject having a neurodevelopmental disorder, the method comprising: a step 11 of acquiring sensor data 14 from one or more wearable sensors 13 configured to be worn by a subject 12, wherein the sensor data comprise motion data, sound data, physiological data, or combinations thereof of the subject, and wherein the sensor data are time-series data; a step 15 of transmitting the sensor data from the one or more wearable sensors to at least one computing device (e.g., a smart device 16); a step 21 of filtering, by the at least one computing device, the sensor data to yield filtered sensor data; a step 22 of processing, by the at least one computing device, the filtered sensor data to yield processed sensor data, wherein the processed sensor data are tabular data; a step 23 of evaluating the sensor data of the subject with respect to target data, wherein the motion data, the sound data, the physiological data, or combinations thereof of the subject are evaluated with respect to target motion data, target sound data, target physiological data, or combinations thereof, respectively; wherein evaluating the sensor data of the subject with respect to the target data comprises providing, by the at least one computing device, an input to an algorithm based on at least one machine learning model that is trained with sensor data, filtered sensor data, processed sensor data, or combinations thereof from the subject and/or with data from at least one additional subject; wherein the algorithm is configured to evaluate the sensor data of the subject with respect to the target data and yield an evaluation score (e.g., prediction score), provide an evaluation score, and compare the evaluation score with a threshold score value; a step 31 of determining, by the at least one computing device, that the sensor data of the subject is equal to or exceed the target data; wherein the computing device monitors and analyzes the evaluation score with respect to the threshold score value to determine that the evaluation score exceeds the threshold score, wherein the evaluation score exceeding the threshold score corresponds to the sensor data exceeding the target data; and a step 40, which is responsive to step 30, of delivering audible sound therapy to the subject, wherein the audible sound therapy comprises a familiar audio sound track, and wherein the audio sound track is repeated at least until it is determined that the threshold score exceeds the evaluation score, wherein the threshold score exceeding the evaluation score corresponds to the target data exceeding the corresponding sensor data of the subject. The method 200 can further comprise a step 45 of optionally informing the subject and/or a caregiver of the sensor data exceeding the target data (e.g., informing of the onset of a pre-meltdown stage). The audible sound therapy may be started by the subject subsequent to the subject being informed of the onset of a pre-meltdown stage. The audible sound therapy may be paused or interrupted by the subject and/or a caregiver, as necessary or desired. In some aspects, the subject may start the audible sound therapy without being informed of the onset of a pre-meltdown stage. In an aspect, the audible sound therapy may have a predetermined volume (e.g., loud, soft, etc.) and volume profile: the volume may either stay substantially constant or change as necessary or desired while the audible sound therapy is delivered. The volume and volume profile during the audible sound therapy may be pre-determined (preset) or may be changed by the subject as necessary or desired during the audible sound therapy. In some aspects, the volume and volume profile during the audible sound therapy may be changed by the caregiver as necessary or desired during the audible sound therapy.

Autism spectrum disorder (ASD) is a complex neurodevelopmental disorder, wherein the diagnosis of ASD is a challenging and elaborate process. However, an early accurate ASD diagnosis was found to be associated with better prognosis, wherein a better prognosis can generally be defined as a better quality of life, ranging from significant gains in cognition, language, and adaptive behavior to more functional outcomes in later life. The social impairment, which is a defining characteristic in subjects (e.g., individuals, patients, etc.) having ASD, correlates with individuals having ASD suffering from an increased incidence of bullying and peer victimization. Additionally, individuals with ASD have a significantly higher risk of self-harm and attempting suicide when compared to the general population, owing in part to the increased incidence of bullying and peer victimization. Further, subjects having ASD can exhibit meltdowns, which can be described as phenomena with varying expressions by which the subjects feel entirely overwhelmed, wherein the meltdowns are sometimes referred to as “socially inappropriate behaviors” or “challenging behaviors.” Nonlimiting examples of meltdown triggers include social demands, frustration (e.g., frustrations over expectations to perform activities and behave within conventional norms), embarrassment, challenges with communication (e.g., failed attempts to be understood, failed attempts to understand the others), emotional triggers, neurological overload, neurological difficulty adjusting to even minor deviations from routine, sensory (e.g., auditory, olfactory, tactile, visual, gustatory) overload, overwhelming aversive sensory stimuli, and the like, or combinations thereof.

Meltdowns add to the list of obstacles to social development, as well as effective education and training of individuals diagnosed with ASD. In addition to the meltdowns being painful experiences for the subjects having ASD, meltdowns are generally socially rejected, thus contributing to the isolation of the subjects having ASD, thereby increasing the risk of self-harm and suicide.

Generally, individuals with ASD may not indicate that they are under stress or experiencing difficulty coping, sometimes owing to the individuals being unaware that they are near a meltdown. Meltdowns can be preceded by a pre-meltdown stage, which is also referred to sometimes as a “rumbling” stage. As would be appreciated by one of skill in the art, and with the help of this disclosure, it is challenging to detect the stress buildup leading to the meltdown phase (e.g., the stress during a pre-meltdown or rumbling stage) by conventional methods. The time duration and the intensity of the pre-meltdown stage varies from one individual to another, as well as from one instance to another for the same individual. However, although the individuals themselves and/or their caregivers may not recognize that a meltdown is impending, meltdowns can present warning signs, wherein such warning signs may be picked up by wearable sensors as disclosed herein. Nonlimiting examples of pre-meltdown behaviors that can be picked up by wearable sensors as disclosed herein include tensing muscles, stimming (e.g., repetitive motions), pacing, kicking, tapping, changing motion frequency, changing motion intensity, changing motion patterns, breathing faster, having an increased heart rate, making louder sounds/noises, and the like, or combinations thereof. In an aspect, the sensor data being equal to or exceeding the target data corresponds to (e.g., correlates with, signals) the onset (e.g., beginning, start) of a pre-meltdown. For example, (i) the motion data of the subject being equal to or exceeding the target motion data; (ii) the sound data of the subject being equal to or exceeding the target sound data; (iii) that the physiological data of the subject being equal to or exceeding the target physiological data; or (iv) any combination of (i)-(iii) corresponds to the onset of a pre-meltdown.

In an aspect, a method for decreasing meltdown incidence and/or severity in a subject having a neurodevelopmental disorder can comprise a step of acquiring sensor data from one or more wearable sensors configured to be worn by a subject, wherein the sensor data comprise motion data, sound data, physiological data, and the like, or combinations thereof of the subject.

In an aspect, the wearable sensor comprises a wearable motion sensor, wherein the wearable motion sensor is configured to acquire motion data of the subject. The wearable motion sensor can comprise a motion sensing unit. The motion sensing unit may comprise a micro-electro-mechanical system (MEMS) based motion sensor, a gyroscope, an accelerometer, a magnetometer, a distance measurement sensor, an absolute position sensor (e.g., a trilateration device), and the like, or combinations thereof.

In an aspect, the wearable sensor comprises a wearable sound sensor, wherein the wearable sound sensor is configured to acquire sound data of the subject. The wearable sound sensor can comprise a microphone, and optionally an amplifier.

In an aspect, the wearable sensor comprises a wearable physiological sensor, wherein the wearable physiological sensor is configured to acquire physiological data of the subject. The wearable physiological sensor can comprise a pulse oximeter, a piezoelectric pressure sensor, a radio frequency identification (RFID) sensor, and the like, or combinations thereof.

Additionally, in an aspect, the wearable sensor comprises one or more sensors configured to detect an environmental or ambient condition. For instance, various environmental stimuli, such as a siren, flashing lights, lightning, thunder, loud music, crowds, or uncomfortable conditions (e.g., extreme ambient conditions) may contribute to the onset of a meltdown or pre-meltdown. For example, in various aspects, the wearable sensor may comprise a sound sensor, a light sensor, temperature sensor, or the like. In some aspects, an environmental sensor may be integrated with a sensor configured to detect various data of the subject. For instance, a sound sensor may be configured to detect both sounds of the subject and ambient noise(s).

In an aspect, a method for decreasing meltdown incidence and/or severity in a subject having a neurodevelopmental disorder can display a system 300, 400 architecture as shown in FIGS. 3 and 4, respectively. The system 300, 400 is configured to monitor subject data (e.g., sensor data of the subject) substantially continuously or semi-continuously, for example by detecting and or sampling data points at a time interval that is less than about 5 minutes (min), alternatively less than about 4 min, alternatively less than about 3 min, alternatively less than about 2 min, alternatively less than about 1 min, alternatively less than about 45 seconds (s), alternatively less than about 30 s, alternatively less than about 15 s, alternatively less than about 10 s, alternatively less than about 5 s, alternatively less than about 4 s, alternatively less than about 3 s, alternatively less than about 2 s, alternatively less than about 1 s, alternatively less than about 750 milliseconds (ms), alternatively less than about 500 ms, alternatively less than about 250 ms, or alternatively less than about 100 ms. The time interval that is sued for sampling the sensor data of the patient may change over time. For example, when the subject appears to be in a steady-state (e.g., habitual state, not in distress, not nearing a pre-meltdown or meltdown stage) the time intervals can be relatively greater. As another example, when the subject appears to be exiting steady-state (e.g., indicating some form of distress, nearing a pre-meltdown or meltdown stage) the time intervals can be relatively shorter. The system 300, 400 can monitor sensor data of the subject by using wearable sensors as disclosed herein, wherein the sensor data of the subject can comprise vital signs, motion, stress, electrocardiogram (ECG) features, electromyogram (EMG) features, and the like, or combinations thereof.

In some aspects, the wearable sensors can be integrated with a wearable item (e.g., wearable device), such as a wrist-mounted wearable sensor device, a fitness tracker, a bracelet device, a wireless-enabled bracelet device, a smartwatch, a fitness watch, a head-mounted wearable device, textile fiber, clothes, elastic bands, and the like, or combinations thereof. In other aspects, the wearable sensors can be directly attached to the human body. In some aspects, the wearable sensors can be integrated with a phone, such as smartphone, wherein the subject keeps the phone on them, for example in a pocket, in their hand, in a phone holder, on a lanyard, etc. In aspects where the wearable sensor is integrated with a phone, the phone can comprise a processor and/or a controller. Further, the device (e.g., wearable item or phone) can comprise speakers for delivering the audible sound therapy. Furthermore, the device (e.g., wearable item or phone) can be connected (e.g., wired or wireless connection) to headphones, earbuds, a speaker, a smart-speaker, etc.

In some aspects, the wearable sensors (e.g., wearable sensors integrated with a wearable device) are configured to connect to a smart device (e.g., smartphone, tablet) via Wi-Fi or Bluetooth. The smart device can comprise a custom-built application (app) as disclosed herein that can receive (e.g., collect) the data from the wearable device. Subsequent to receiving the sensor data, the app can manipulate the sensor data (e.g., evaluate sensor data, filter sensor data, process sensor data, apply machine learning, apply an algorithm, etc.) locally on the smart device (e.g., edge computing) and/or remotely by transmitting the data to a cloud platform (e.g., cloud computing).

In an aspect, a method for decreasing meltdown incidence and/or severity in a subject having a neurodevelopmental disorder can comprise a step of comparing the sensor data of the subject with target data, wherein the motion data, the sound data, the physiological data, and the like, or combinations thereof of the subject are compared with target motion data, target sound data, target physiological data, and the like, or combinations thereof, respectively. In such aspect, the sensor data being equal to or exceeding target data correlates with the onset of a pre-meltdown stage for the subject.

In an aspect, the wearable sensor can be integrated with a control system, wherein the control system receives the sensor data from the one or more wearable sensors; wherein the control system comprises at least one processor and at least one controller; wherein the at least one processor compares the sensor data to the target data; wherein, when at least one of the sensor data is equal to or exceed the corresponding target data, the at least one processor signals the at least one controller; and wherein the at least one controller delivers/the audible sound therapy to the subject. In some aspects, the control system comprises a mobile device, wherein the mobile device can be selected from the group consisting of a smartphone, a smartwatch, a tablet, a laptop, a personal computer, and combinations thereof. The wearable sensor can be connected with said mobile device over a wired or wireless data connection. In an aspect, the wearable sensor can be integrated in a wearable computing or communication device, such as a smartphone, smartwatch or other watch or wristband (e.g., fitness tracker) configurable to be connected (e.g., via Bluetooth) to a smartphone, tablet, laptop, computer, and the like, or combinations thereof. In some aspects, the wearable sensors can be integrated with a smartphone and/or a smartwatch, wherein the smartphone and/or the smartwatch comprise the wearable sensors, the processor, the controller, and optionally the speakers. In an aspect, the control system provides for real-time delivery of the audible sound therapy to the subject. In an aspect, the audible sound therapy can be delivered to the subject via speakers, earbuds, or headphones.

In an aspect, evaluating the sensor data of the subject with respect to the target data can provide for substantially real-time or near real-time feedback to the subject in the form of audible sound music therapy. The term “near real-time,” as used herein, refers to a delay that is introduced by sensor data manipulation (e.g., transmitting, filtering, processing, inputting, computing, etc.) between the occurrence of an event (e.g., a live event, such as a change in the sensor data of the subject indicating the onset of a pre-meltdown) and the use of the information derived from manipulating the sensor data associated with the event (e.g., the delivery of the audible sound therapy to the subject). For example, classifying an event as a near real-time event refers to the delay that allows the use of the information derived from the sensor data near the time of the live event, wherein such delay refers to the difference between the real-time event occurrence and the use of the information derived from manipulating the sensor data associated with the event (e.g., the delivery of the audible sound therapy to the subject). For example, the difference between the real-time event occurrence and the use of the information derived from manipulating the sensor data associated with the event (e.g., the delivery of the audible sound therapy to the subject) can be less than about 5 min, alternatively less than about 4 min, alternatively less than about 3 min, alternatively less than about 2 min, alternatively less than about 1 min, alternatively less than about 45 s, alternatively less than about 30 s, alternatively less than about 15 s, alternatively less than about 10 s, alternatively less than about 5 s, alternatively less than about 4 s, alternatively less than about 3 s, alternatively less than about 2 s, alternatively less than about 1 s, alternatively less than about 750 ms, alternatively less than about 500 ms, alternatively less than about 250 ms, or alternatively less than about 100 ms.

In an aspect, the target data can be personalized (e.g., calibrated, fine-tuned) for each the subject. In other words, the target data (e.g., target motion data, target sound data, target physiological data) is individually determined based on the subject's usual (e.g., habitual, ordinary) motions, motion frequency, motion intensity, motion patterns, sounds, sound frequency, sound patterns, and physiology. For individuals with ASD, usual motions may differ significantly from one individual to another, as individuals with ASD habitually engage in stimming (e.g., repetitive motions), and thus what is target motion data for an individual with ASD may be habitual stimming for another individual with ASD.

In an aspect, a method for decreasing meltdown incidence and/or severity in a subject having a neurodevelopmental disorder can comprise, when it is determined that the sensor data are equal to or exceed a corresponding target data and/or deviate from an “ordinary” (e.g., habitual, usual) profile associated with a subject, a step of delivering audible sound therapy to the subject, wherein the audible sound therapy comprises an audio sound track, wherein the audio sound track is characterized by a track rhythm and by a track beat, wherein the audio sound track is familiar to the subject, and wherein the audio sound track is repeated at least until it is determined, in any sequence, at least one of the following: that the target motion data exceed the motion data of the subject; that the target sound data exceed the sound data of the subject; and that the target physiological data exceed the physiological data of the subject. The audio sound data, for example, audio sound data that is “familiar” to the subject, may be particularly associated with the subject, for example, based upon a determination that the audio sound data is effective to elicit a response (e.g., a physiological response) in the subject, for instance, a determination that the audio sound data (e.g., the audio sound track) is effective to prevent onset of a meltdown stage for the subject or decrease severity of a meltdown stage for the subject. The audio sound track can comprise a song, a music album, an audio book chapter, an audio book, a recited poem, a collection of recited poems, and the like, or combinations thereof. The audio sound track is familiar to the subject, e.g., the subject has heard the audio sound track previously on more than one occasion, and the audio sound track has consistently (e.g., on two or more distinct occasions) displayed a calming or soothing effect (as opposed to an agitating effect) on the subject.

In an aspect, the control system selects the audio sound track that is familiar to the subject (e.g., associated with the subject based upon a determination that the audio sound data is effective to elicit a response, for example, to prevent onset of a meltdown stage for the subject or decrease severity of a meltdown stage for the subject) from a library of audio sound tracks that are familiar to the subject; wherein said selection is based on the type of sensor data (e.g., motion data, physiological data, sound data, or combinations thereof) of the subject that is equal to or exceed the corresponding target data and/or the magnitude of difference between the sensor data of the subject and the corresponding target data. For example, when the subject is only starting to get agitated, a first familiar audio sound track may be played for the subject. As another example, when the subject is mildly agitated, a second familiar audio sound track may be played for the subject. As yet another example, when the subject is clearly agitated, a third familiar audio sound track may be played for the subject. Each of the audio tracks (e.g., first, second and third audio tracks) can be selected based on a previous known response of the subject to each individual audio track. While a caregiver may help indicate what audio track may be helpful in a specific state of agitation for the subject, the method for decreasing meltdown incidence and/or severity in a subject having a neurodevelopmental disorder excludes an input from a caregiver for delivering the audible sound therapy to the subject. In other words, data transmission from the sensors to the processor, selection by the processor of a specific audio track to be delivered as audible sound therapy to the subject, communication between processor and controller, delivery by the controller of audible sound therapy to the subject, or combinations thereof do not require an input from the caregiver or a clinician.

In an aspect, the audible sound therapy comprises a single audio sound track that may be repeated as necessary. In another aspect, the audible sound therapy may comprise two or more audio sound tracks that may be repeated as necessary and in any suitable order. For example, the audible sound therapy may comprise a music album that may be played in order or shuffled, wherein each track and/or the entire album may be repeated as necessary.

In an aspect, delivering audible sound therapy to the subject prevents the onset of a meltdown stage for the subject or decreases the severity of a meltdown stage for the subject. The wearable sensors can provide for recognizing the period preceding a meltdown state (e.g., pre-meltdown or rumbling stage characterized by increased agitation, which can be physical, mental, or emotional), and as a consequence of the sensor transmitting data to the processor, the processor may signal the controller to play favorite music (e.g., a favorite song on repeat) during the rumbling stage, wherein the meltdown is minimized or eliminated.

In an aspect, the wearable sensor can comprise a motion sensor (e.g., a sensor configured to detect motion data), wherein the motion sensor transmits motion data to the processor. The motion data comprise motion frequency and/or motion intensity; wherein the target motion data comprise target motion frequency and/or motion intensity, respectively; and wherein the audible sound therapy is delivered to the subject at least until the target motion frequency and/or target motion intensity exceed the motion frequency and/or motion intensity, respectively, of the subject. For purposes of the disclosure herein, the motion frequency of an individual refers to how quickly (or slowly) the subject moves, wherein the subject can be stimming, walking, pacing, running, spinning taping, swinging, or otherwise engaging in motion activities (whether the motion is repetitive or not). For purposes of the disclosure herein, the motion frequency can be defined by the amount or level of continuous motion of a subject per a defined unit of time (e.g., continuous motions per minute). Further, for purposes of the disclosure herein, a continuous motion can be a motion that is made substantially in about the same direction, and once the direction changes, another continuous motion starts. In other words, breaks or interruptions in motions, as well as changes in the directionality of the motions separate one continuous motion from another. While walking in a single direction can be thought of as a continuous motion for the whole body, during walking the limbs or portions thereof engage in motions that may alternate directions or have a break in the motion, thereby defining several continuous motions as disclosed herein. For purposes of the disclosure herein, the motion intensity of an individual refers to the magnitude (e.g., power, force, etc.) of motion with which the subject moves, wherein the subject can be stimming, walking, pacing, running, spinning, tapping, swinging, or otherwise engaging in motion activities (whether the motion is repetitive or not). Without wishing to be limited by theory, the motion magnitude correlates with the size and the speed of the subject that is in motion. For example, a subject who is running would have a greater motion intensity than a subject who is walking over the same distance. Generally, a subject that moves faster will have a greater motion intensity or motion magnitude.

In an aspect, the track rhythm and/or the track beat of the audio sound track that is familiar to the subject corresponds to a motion frequency of the subject that is less than the target motion frequency.

In an aspect, when a subject is determined to have a motion frequency and/or motion intensity that is equal to or exceeds the target motion frequency and/or target motion intensity, respectively, the audible sound therapy can be delivered to the subject, wherein the subject achieves a motion frequency and/or motion intensity that is less than the target motion frequency and/or target motion intensity, respectively, as a result of the audible sound therapy. For example, this can be thought of as the motions (e.g., repetitive motions, such as stimming) of the subject substantially synchronizing into the rhythm of a favorite song and stabilizing there instead of growing out of control. Further, as the audible sound therapy continues (e.g., the audible sound track keeps repeating) to be delivered to the subject, the stimming of the subject, as well as the agitation associated therewith would further decrease (e.g., dissipate, diminish) and/or return to consistent habitual stimming as the music plays (e.g., the song keeps repeating). In such aspect, the track rhythm and/or the track beat can provide for the subject achieving a rhythmic motion that is substantially synchronized to the track rhythm and/or to the track beat, wherein the target motion frequency and/or target motion intensity exceed the motion frequency and/or motion intensity, respectively, of the subject. In some aspects, the rhythmic motion can comprise a body motion, a torso motion, a limb motion, an arm motion, a hand motion, a finger motion, a leg motion, a foot motion, a toe motion, a knee motion, a head motion, and the like, or combinations thereof. For example, the subject may engage in swinging, swaying, dancing, performing dance-like motions, and the like, or combinations thereof as a result of listening to a favorite song, thereby resulting in the meltdowns being prevented in some instances, or at least have their severity decreased.

In some aspects, the motion data can comprise motion pattern data, wherein the target motion data comprise habitual motion pattern data; and wherein the audible sound therapy is delivered to the subject at least until the habitual motion pattern data are substantially the same as the motion pattern data of the subject. In such aspect, the motion data being equal to or exceeding a corresponding target motion data refers to (e.g., is substantially equivalent to) the motion pattern data of the subject being different from the habitual motion pattern data.

In an aspect, when a subject is determined to have a motion pattern that is different from the habitual motion pattern, the audible sound therapy can be delivered to the subject, wherein the subject achieves a motion pattern that is substantially the same as the habitual motion pattern as a result of the audible sound therapy.

In an aspect, the wearable sensor can comprise a physiological sensor (e.g., a sensor configured to detect physiological data), wherein the physiological sensor transmits physiological data to the processor. In some aspects, the physiological data can comprise heart rate, blood pressure, respiration rate, breathing pattern, oxygen saturation rate, muscle tension level, temperature, one or more electrocardiogram (ECG) features, one or more electromyogram (EMG) features, and the like, or combinations thereof. The physiological data can help determine when an individual is experiencing an increased state of agitation, stress, distress, etc. In some instances, individuals in a pre-meltdown stage can exhibit repetitive thoughts that have a frequency that is increased when compared to the frequency of such thoughts during habitual repetitive thoughts, and such repetitive thoughts may trigger a change in the physiological data of the subject, for example when the repetitive thoughts occur with an increased frequency.

In an aspect, when a subject is determined to have an increased state of agitation based on physiological data transmitted from the physiological sensor to the processor, the audible sound therapy can be delivered to the subject at least until the target physiological data exceed the physiological data of the subject. For example, when the subject has an agitation state that is accompanied by repetitive thoughts cycling with increased frequency, the audible sound therapy can be delivered to the subject, wherein the subject achieves a repetitive thought frequency that is less than a target repetitive thought frequency as a result of the audible sound therapy. For example, this can be thought of as the repetitive thoughts of the subject substantially synchronizing into the rhythm of a favorite song and stabilizing there instead of growing out of control. Further, as the audible sound therapy continues (e.g., the audible sound track keeps repeating) to be delivered to the subject, the repetitive thoughts of the subject, as well as the agitation associated therewith would further decrease (e.g., dissipate, diminish) and/or return to consistent habitual repetitive thinking as the music plays (e.g., the song keeps repeating), thereby resulting in the meltdowns being prevented in some instances, or at least have their severity decreased.

In an aspect, the wearable sensor can comprise a sound sensor (e.g., a sensor configured to detect sound data), wherein the sound sensor transmits sound data to the processor, and wherein the sound data comprise vocal sounds produced by the subject. The audible sound therapy can be delivered to the subject at least until the target sound data exceed the sound data of the subject. For example, when the subject has an agitation state that is accompanied by vocal sounds produced by the subject with increased volume and/or frequency, the audible sound therapy can be delivered to the subject, wherein the subject achieves a vocal sound volume and/or frequency that is less than a target vocal sound volume and/or frequency as a result of the audible sound therapy. For example, this can be thought of as the vocal sound volume and/or frequency of the subject substantially synchronizing into the volume and/or rhythm, respectively of a favorite song and stabilizing there instead of growing out of control. Further, as the audible sound therapy continues (e.g., the audible sound track keeps repeating) to be delivered to the subject, the vocal sound volume and/or frequency of the subject, as well as the agitation associated therewith would further decrease (e.g., dissipate, diminish) and/or return to consistent habitual vocal sound volume and/or frequency as the music plays (e.g., the song keeps repeating). For example, the subject may engage in singing, humming, rhythmically talking, and the like, or combinations thereof (for example along with the audio sound track) as a result of listening to a favorite song, thereby resulting in the meltdowns being prevented in some instances, or at least have their severity decreased.

In some aspects, the sound data can comprise sound pattern data (e.g., pattern of vocal sounds produced by the subject), wherein the target sound data comprise habitual sound pattern data; and wherein the audible sound therapy is delivered to the subject at least until the habitual sound pattern data are substantially the same as the sound pattern data of the subject. In such aspect, the sound data being equal to or exceeding a corresponding target sound data refers to (e.g., is substantially equivalent to) the sound pattern data of the subject being different from the habitual sound pattern data.

In an aspect, when a subject is determined to have a sound pattern that is different from the habitual sound pattern, the audible sound therapy can be delivered to the subject, wherein the subject achieves a sound pattern that is substantially the same as the habitual sound pattern as a result of the audible sound therapy.

In some aspects, the wearable sensors may detect and transmit only motion data. In other aspects, the wearable sensors may detect and transmit only physiological data. In yet other aspects, the wearable sensors may detect and transmit only sound data. In still yet other aspects, the wearable sensors may detect and transmit both motion data and physiological data. In still yet other aspects, the wearable sensors may detect and transmit both sound data and physiological data. In still yet other aspects, the wearable sensors may detect and transmit both motion data and sound data. In still yet other aspects, the wearable sensors may detect and transmit motion data, physiological data, and sound data.

In an aspect, a system (e.g., system 300 in FIG. 3) can comprise one or more wearable sensors 13 configured to detect sensor data 14 of a subject 12; wherein the one or more wearable sensors 13 comprise at least one sensor configured to detect motion data, at least one sensor configured to detect sound data, at least one sensor configured to detect physiological data, or combinations thereof; and a control system configured to receive the sensor data 14 of the subject 12 from the one or more wearable sensors 13; wherein the control system comprises at least one processor and at least one controller; wherein the at least one processor compares the sensor data 14 to target data; wherein, when at least one of the sensor data are equal to or exceed 30 the target data, the at least one processor is configured to signal the at least one controller; and wherein the at least one controller delivers 40 an audible sound therapy to the subject 12; wherein the audible sound therapy comprises an audio sound track, wherein the audio sound track is characterized by a track rhythm and by a track beat, wherein the audio sound track is familiar to the subject 12, and wherein the audio sound track is repeated at least until it is determined that the target data exceed the sensor data of the subject 12. In such aspect, the at least one processor compares the motion data, the sound data, the physiological data, or combinations thereof of the subject with target motion data, target sound data, target physiological data, or combinations thereof, respectively. In such aspect, the controller can comprise (i) an algorithm based on at least one machine learning model that is trained with sensor data 14 from the subject 12 and/or with data from at least one additional subject, wherein the algorithm is configured to evaluate 20 the sensor data 14 of the subject 12 with respect to target data; and (ii) a non-transitory computer readable medium that stores instructions that when executed by the processor, causes the processor to: receive, using the control system, an input comprising sensor data of the subject; provide, by the control system, the input to the algorithm; determine, by the control system, an evaluation result comprising an indication that the sensor data of the subject is equal to or exceed 30 the target data by using the algorithm; wherein the indication that the sensor data of the subject is equal to or exceed the target data is an evaluation score being equal to or greater than a threshold score value; and deliver 40, by the control system, audible sound therapy to the subject, wherein the audible sound therapy comprises an audio sound track, wherein the audio sound track is familiar to the subject, and wherein the audio sound track is configured to be repeated at least until the target data exceed the sensor data of the subject.

In an aspect, a system (e.g., system 400 in FIG. 4) can comprise one or more wearable sensors 13 configured to detect sensor data 14 of a subject 12; wherein the one or more wearable sensors 13 comprise at least one sensor configured to detect motion data, at least one sensor configured to detect sound data, at least one sensor configured to detect physiological data, or combinations thereof; and a computing system configured to receive 15 the sensor data 14 of the subject 12 from the one or more wearable sensors 13; wherein the computing system comprises (i) at least one processor; (ii) an algorithm based on at least one machine learning model that is trained with sensor data 14 from the subject 12 and/or with data from at least one additional subject, wherein the algorithm is configured to evaluate 23 the sensor data of the subject with respect to target data, provide an evaluation score, and compare the evaluation score with a threshold score value; and (iii) a non-transitory computer readable medium that stores instructions that when executed by the processor, causes the processor to: receive, using the computing system, an input comprising sensor data of the subject; provide, by the computing system, the input to the algorithm; and determine, by the computing system, an evaluation result comprising an indication that the sensor data of the subject is equal to or exceed the target data by using the algorithm; wherein the indication that the sensor data of the subject is equal to or exceed the target data is the evaluation score being equal to or greater than the threshold score value 31; wherein the sensor data being equal to or exceeding target data correlates with the onset of a pre-meltdown stage for the subject. In such aspect, the processor is configured to deliver 40, by the computing system, an audible sound therapy to the subject; wherein the audible sound therapy comprises an audio sound track, wherein the audio sound track is characterized by a track rhythm and by a track beat, wherein the audio sound track is familiar to the subject, and wherein the audio sound track is repeated at least until it is determined, by the computing system, that the threshold score value exceeds the evaluation score. In such aspect, the sensor data 14 are time-series data; wherein the computing system is configured to filter the sensor data to yield filtered sensor data; wherein the computing system is configured to process the filtered sensor data to yield processed sensor data, wherein the processed sensor data are tabular data; and wherein the input to the algorithm comprises tabular data. In such aspect, the sensor data 14 can be manipulated (e.g., filtered, processed, evaluated, etc.), by the at least one computing device, via edge computing, via cloud computing, or via both edge computing and cloud computing.

Generally, edge computing refers to utilizing compute resources present in a smart device (e.g., smart device that receives the sensor data from the wearable sensors). In some aspects, at least a portion of the computation employed in the method for decreasing meltdown incidence and/or severity in a subject having a neurodevelopmental disorder as disclosed herein can comprise edge computing. For example, the smart device can employ edge computing to filter sensor data, clean sensor data, remove noise from the sensor data, remove background noise from the sensor data, extract features from sensor data, process the sensor data, convert the sensor data to tabular data (e.g., from time-series data to tabular data), pass the sensor data (e.g., filtered and/or processed sensor data) through a machine learning model (e.g., a machine learning model that may be integrated with a custom application as disclosed herein), and the like, or combinations thereof. The machine learning model running on the smart device can then output a score (e.g., an evaluation score, a prediction score) in between 0 and 1. The evaluation score can be periodically monitored on the smart device by the custom application as disclosed herein (e.g., the evaluation score can be compared with a threshold score value). In aspects where the evaluation score crosses a predetermined threshold (e.g., threshold score value), the custom application can deliver the audible sound therapy (e.g., play the music from a pre-prepared playlist). The audible sound therapy may continue to be delivered (e.g., music may continue to play) by the custom application on the smart device at least until the evaluation score returns to below the threshold score value. Optionally, the subject and/or the caregiver may be notified by the custom application on the smart device of the potential onset of pre-meltdown. In some aspects, the data collected from the app can be backed up in the cloud periodically. In such aspects, the data to be backed up can be encrypted, wherein the encrypted data can be transported over a secure file transfer protocol in order to prevent data breach in-transit (e.g., over network such as Wi-Fi) and/or at-rest (e.g., in the cloud).

In an aspect, computing locally on the smart device may advantageously display relatively low latency and/or relatively high security, for example because the data do not need to be transported to a distant location over a network.

Generally, cloud computing can encompass utilizing compute resources from a cloud service provider (e.g., Amazon Web Services (AWS), Google Cloud Platforms (GCP), Microsoft Azure, etc.). In some aspects, at least a portion of the computation employed in the method for decreasing meltdown incidence and/or severity in a subject having a neurodevelopmental disorder as disclosed herein can comprise cloud computing. The sensor data collected by the custom application as disclosed herein can be encrypted and transferred over secure file transfer protocol to the cloud, to prevent data breach in-transit (e.g., over network such as Wi-Fi) and/or at-rest (e.g., in the cloud). Once the data reaches the cloud, the data can be manipulated to filter sensor data, clean sensor data, remove noise from the sensor data, remove background noise from the sensor data, extract features from sensor data, process the sensor data, convert the sensor data to tabular data (e.g., from time-series data to tabular data), pass the sensor data (e.g., filtered and/or processed sensor data) through a machine learning model (e.g., a machine learning model that may be integrated with a custom application as disclosed herein), and the like, or combinations thereof. The machine learning model running in the cloud can then output a score (e.g., an evaluation score) in between 0 and 1. The evaluation score can be periodically monitored in the cloud (e.g., the evaluation score can be compared with a threshold score value). In aspects where the evaluation score crosses a predetermined threshold (e.g., threshold score value), the cloud platform can deliver the audible sound therapy (e.g., play the music from a pre-prepared playlist). The audible sound therapy may continue to be delivered (e.g., music may continue to play) by the cloud platform at least until the evaluation score returns to below the threshold score value. Optionally, the subject and/or the caregiver may be notified by the cloud platform of the potential onset of pre-meltdown.

In an aspect, computing on the cloud may advantageously provide an ability to employ for sensor data collection a relatively wide range of devices that may not have enough compute resources locally, while displaying relatively faster processing and/or prediction time.

In some aspects, the local smart device and cloud platform can be employed in a hybrid environment, wherein the data cleaning (e.g., filtering) and processing can be done locally on the smart device, and wherein the processed data can be encrypted and transmitted to the cloud to apply machine learning and continuously monitor the output of the algorithm.

In an aspect, the data (e.g., sensor data received from the wearable sensor) may be processed and run through the machine learning model at variable length (e.g., intervals) depending on the need and compute resources. The frequency of sampling the data and/or running the data through the machine learning model may be a frequently effective to detect the onset of a pre-meltdown. In other words, data may be sampled and run through the machine learning often enough (e.g., every about 5 min, alternatively about 4 min, alternatively about 3 min, alternatively about 2 min, alternatively about 1 min, alternatively about 45 s, alternatively about 30 s, alternatively about 15 s, alternatively about 10 s, alternatively about 5 s, alternatively about 4 s, alternatively about 3 s, alternatively about 2 s, alternatively about 1 s, alternatively about 750 ms, alternatively about 500 ms, alternatively about 250 ms, or alternatively about 100 ms) as to not miss the pre-meltdown, and additionally to have the time necessary to administer the music therapy.

In some aspects, the sensor data may be compared with the target data without the use of a machine learning model, for example through mathematical computations that exclude the use of a machine learning model.

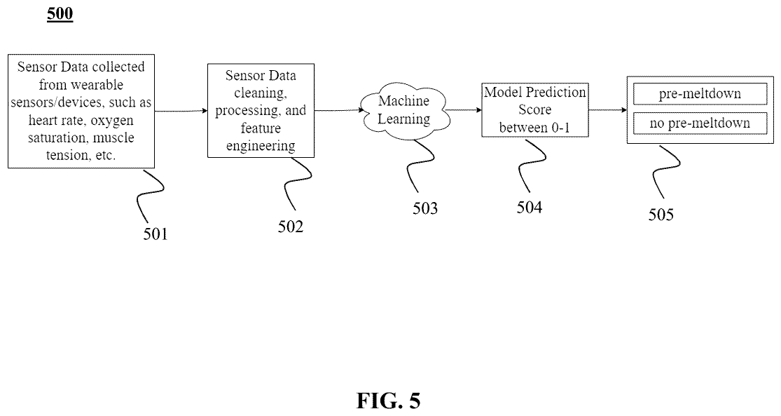

In other aspects, the sensor data may be compared with the target data by using a machine learning model 500, for example as displayed in FIG. 5. The machine learning model can aggregate information from multiple features (e.g., different types of sensor data and/or data features 501, which may be subjected to cleaning, processing, and feature engineering 502), wherein the machine learning model 503 can provide a single number or score 504 (e.g., evaluation score) in between 0 and 1, wherein the evaluation score is a probability of a condition (e.g., pre-meltdown) as occurring or not occurring 505. As would be appreciated by one of skill in the art, and with the help of this disclosure, the actual discrimination between different classes can be done based on a threshold score value, wherein the threshold score value is between 0 and 1, which is the basis for discriminating between a positive class (e.g., exceeds threshold, greater than the threshold) and a negative class (e.g., does not exceed threshold, less than the threshold). When the machine learning model yields an evaluation score that is equal to or greater than the threshold score value for a given subject at a given time, then the class is a positive class (e.g., it signals the need to deliver the audible music therapy). When the machine learning model yields an evaluation score that is less than the threshold score value for a given subject at a given time, then the class is a negative class (e.g., it signals that there is no need to deliver the audible music therapy).

In an aspect, developing a machine learning model for the method for decreasing meltdown incidence and/or severity in a subject having a neurodevelopmental disorder as disclosed herein can comprise acquiring labeled training data. For purposes of the disclosure herein, the term “label” with respect to data (e.g., training data) refers to the subject having experienced a pre-meltdown (or meltdown) or not, and in the case where a pre-meltdown (or meltdown) was experienced, at what point in time did the subject experience the pre-meltdown (or meltdown). The timing component can be relevant while building the machine learning model, because the model is to be trained to be effective to predict the onset of pre-meltdown far enough in advance that necessary actions, such as audible sound therapy (e.g., sound/music therapy) can be administered. In some aspects, data may be labelled by using information provided by the subject and/or a caregiver (e.g., a pre-meltdown (or meltdown) may be self-reported by the subject and/or by a caregiver). In other aspects, data may be labelled by a healthcare professional based on subject retrospective data (e.g., a healthcare professional may identify and label a pre-meltdown (or meltdown) based on subject retrospective data). In yet other aspects, data may be labelled by using information provided by the subject and/or a caregiver, and by a healthcare professional based on subject retrospective data. Information about the duration and severity of the pre-meltdown (or meltdown) may also be collected. The time at which the pre-meltdown (or meltdown) starts to occur may be referred to as the onset time for purposes of the disclosure herein.

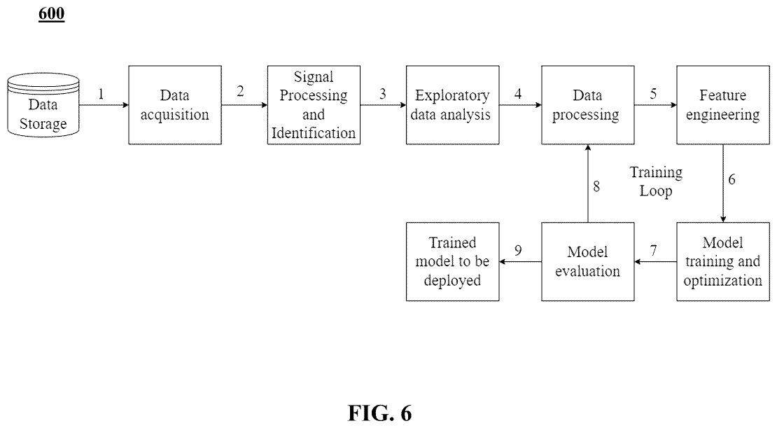

In an aspect, a machine learning model as disclosed herein can be trained 600 as displayed in FIG. 6. As disclosed herein, the machine learning task comprises the prediction the onset of a pre-meltdown in a subject having ASD based on wearable sensor data (e.g., motion data, physiological data, sound data, etc.), collected via wearable sensors, which in some aspects may be integrated with wearable devices. In an aspect, the machine learning model as disclosed herein comprises predicting the onset of a pre-meltdown with sufficient time in advance so that necessary interventions can be implemented, such as in the form of delivering audible sound therapy. In an aspect, the machine learning model as disclosed herein can yield predictions substantially continuously in real-time, for example by utilizing historical data available up until the time of the prediction or a portion of such available historical data. For purposes of the disclosure herein, the time at which the machine learning model makes a prediction can be referred to as “prediction time.” In an aspect, the frequency of prediction by the machine learning model may be substantially constant or may be variable.

In an aspect, prior to data processing and feature engineering to isolate data for input to the machine learning model, the data acquired from the wearable sensor can be manipulated in order to filter noise and/or isolate the meltdown signal for each feature variable. The signal manipulation and identification, which is referred to as data “filtering” for purposes of the disclosure herein, can provide for (i) noise (e.g., background noise) removal from the sensor data, wherein the noise may interfere with data extracted for the machine learning model, and/or (ii) signal isolation associated with pre-meltdowns and filtering data from a non-meltdown activity that may present sensor data similar to the sensor data associated with pre-meltdowns.

In an aspect, one or more digital signal processing (DSP) filters can be constructed by examining the signals (e.g., sensor data) during model development. From the data collected during model development, the magnitude and frequency ranges of the data can be determined, in order to identify the appropriate data frequencies to amplify/pass through the filter (data passband) and the signal frequencies to attenuate (stopband). The stopband frequencies can include the frequency ranges of external noise (e.g., electrical noise, background movement levels, and other relevant high frequency noise) and/or signals associated with other relevant activity (e.g., exercise, sleeping, eating, etc.) that may comprise non-meltdown activities.

In an aspect, the set of passband and stopband frequencies can provide for a series of passive, active, and/or adaptive filters that can be utilized to isolate the pre-meltdown signal. The passive and active filters can include band-pass filters with a stopband that filters (i) high frequency noise due to electrical and/or other interference and (ii) low frequency signals from steady repetitive signals or other non-meltdown signals. The adaptive filters can be implemented to capture pre-meltdowns vs. other signals, as well as monitor how pre-meltdowns change over time by utilizing the pre-meltdown data in combination with other signals and adaptively changing the filter pattern to isolate the pre-meltdown signal. Individual filters can be utilized for each of the distinct features which are being captured by the wearable sensor. The type of wearable sensor can generally determine the extent of filtering necessary in order to optimize performance and minimize latency of filtering to model prediction. The filtered signal (e.g., filtered sensor data) can be further processed to yield processed sensor data as disclosed herein, wherein the processed sensor data can be input to a machine learning algorithm.

In an aspect, the wearable sensor can provide for time-series data, e.g., the wearable sensor gathers time-series data. In other words, the observations (e.g., sensor data) are collected through repeated measurements over time. For example, and in the context of a wearable sensor, an input that may be tracked over time comprises heart rate. The sensor data (e.g., heart rate data) for training can be acquired from a storage device (e.g., smart device and/or cloud storage). At training time, the time-series data can be data collected from a large number of training examples (e.g., collected from a large dataset of pre-meltdown and non-meltdown data), tracked through the wearable sensor or device. Subsequent to filtering, this dataset (e.g., training dataset) can undergo exploratory data analysis to understand the structure/distribution and/or quality of the dataset. Then, the dataset can be corrected or reduced to produce a corrected dataset which excludes outlier data and/or data providing for substantial missingness that may skew the results.

In an aspect, the corrected dataset can be further manipulated; for example by imputing extreme outliers and/or unidentified characters, and/or removing features that may be highly correlated with other non-meltdown events, have a lot of missing values, and/or are relatively non-important.

The time-series data can be subjected to feature extraction. In some aspects, the time-series data may be condensed in the form of summary statistics including features such as minimum, maximum, mean, median, standard deviation, most recent measurement, and the like, or combinations thereof. In other aspects, the time-series data may be aggregated in bins over time (e.g., averaged every about 2 minutes of heart rate). In yet other aspects, the time-series data can be condensed and aggregated in bins over time. In aspects where the time-series data is both condensed and aggregated in bins over time, multiple input features can be constructed from each sensor data type collected from the wearable, thereby increasing or maximizing the amount of information extracted for the model training. The resulting data can be stored in the form of tabular data, which can then be passed through machine learning algorithms. As would be appreciated by one of skill in the art, and with the help of this disclosure, the machine learning model can use sensor data up until the time of onset of pre-meltdown, in order to prevent data leakage. The machine learning model may take substantially all of the historical data up until the time of onset of pre-meltdown or a portion thereof (e.g., a subset of the historical data which may be more relevant to the onset time).

In an aspect, data for negative class examples (e.g., non-meltdown data) can be gathered by processing data from the subject having ASD, for example data from when the subject did not experience a meltdown and when they were performing other activities. As would be appreciated by one of skill in the art, and with the help of this disclosure, the subject that provides non-meltdown data can experience a pre-meltdown at a later time. For example, subject A may experience a pre-meltdown at 1 P.M. on day X. However, data for subject A at 10 A.M. on day X can be labeled as “non-meltdown data” provided that subject A was in a non-meltdown time period at 10 A.M. on day X, as that time is sufficiently in the past in relation to the pre-meltdown at 1 P.M. on day X (provided that no pre-meltdown occurs between 10 A.M. and 1 P.M. on day X). The determination of “sufficiently in the past” can be made based on healthcare provider expert opinion and/or data analysis. For example, “sufficiently in the past” can be equal to or greater than 15 min, 30 min, 1 h, 2 h, 3 h, 4 h, 5 h, 6 h, 7 h, 8 h, 9 h, 10 h, or more. In some aspects, the subject may provide data for the positive class. In other aspects, the subject may provide data for the positive class. In yet other aspects, the same subject may provide data for both the positive class and the negative class.

In an aspect, subsequent to the featurization step, the data can be split into a training data set, a validation data set, and a testing data set with a random stratified split, such that there is substantially no overlap between the datasets (e.g., in terms of data points in time, in terms of subjects, etc.). The random stratified split can provide for the proportion of positive (pre-meltdown) and negative (non-meltdown) classes being substantially equivalent. The training set can comprise a set of data points can provide for teaching the machine learning model as disclosed herein to differentiate between a subject that may experience an onset of ASD pre-meltdown or may not experience an onset of ASD pre-meltdown. The validation dataset can provide for optimizing parameters and hyperparameters of the model. Generally, machine learning models can choose from a wide range of parameter values, wherein model parameters refer to the values which control the way model inputs are transformed into the model outputs, and wherein hyperparameters refer to the values which control the structure and construction of the model. As would be appreciated by one of skill in the art, and with the help of this disclosure, a machine learning model may be initialized with a certain set of parameters and hyperparameters, wherein the set of parameters and hyperparameters may be changed during the course of training in order to provide optimal results in the validation dataset, which is known to the one of skill in the art as parameter/hyperparameter optimization. Subsequent to model optimization, the model can be tested on a testing dataset (e.g., final testing dataset) to produce testing set results (e.g., final set of results).

In an aspect, the output of a machine learning model (e.g., evaluation score) can have a value in between 0 and 1. As would be appreciated by one of skill in the art, and with the help of this disclosure, the evaluation score value in itself does not provide information about whether the model is predicting one class versus another class (e.g., positive class versus negative class). A threshold (e.g., threshold score, threshold score value) can be selected, wherein an evaluation score that is equal to or greater than the threshold score value is considered as a positive output (e.g., a positive class), and wherein an evaluation score that is less than the threshold score value is considered as a negative output (e.g., a negative class). For example, with a threshold of 0.4, a model value (e.g., evaluation score) of 0.5 is classified as positive. As another example, with a threshold value of 0.6, a model value (e.g., evaluation score) of 0.5 is classified as negative.

Nonlimiting examples of machine learning models suitable for use in the present disclosure include a deep learning model, a recurrent neural networks model, a classical tree-based machine learning model, a decision tree type model, a regression type model, and the like, or combinations thereof. Generally, different machine learning models can be trained and evaluated during the course of building an algorithm, wherein one or more machine learning models can be selected, based on the performance of the model (e.g., best performing models can be selected).

In an aspect, the method that for decreasing meltdown incidence and/or severity in a subject having a neurodevelopmental disorder as disclosed herein can comprise deploying the trained machine learning model. During machine learning model deployment (e.g., the model is deployed and the algorithm is running in real-time evaluating sensor data from a subject having ASD with respect to target data), the wearable sensor (e.g., which may be integrated with a wearable device) substantially continuously collects the sensor data from the subject having ASD. Once the machine learning model is deployed, the sensor data acquired from the subject can undergo substantially similar manipulation as the data used for training the model, e.g., sensor data clearing, processing, and feature extraction steps that were used during the training time and passed through the trained machine learning model. The deployed model can then output an evaluation score in between 0 and 1.

In an aspect, the performance of the trained model may be evaluated based on performance metrics, wherein the performance metrics can comprise (i) confusion matrices displaying the number of true positives, false positives, true negatives, and false negatives predicted by the model; (ii) area under the receiver operator characteristic (AUROC) curve which plots true positive rate (TPR, sensitivity, or recall) vs. false positive rate (FPR or 1-specificity) and computes the area under this curve; (iii) statistics for binary classification; or (iv) any combination of (i)-(iii). Nonlimiting examples of statistics for binary classification include sensitivity, specificity, positive predictive value (PPV or precision), negative predictive value (NPV), diagnostic odds ratio (DOR), positive likelihood ratio (LR+), negative likelihood ratio (LR−), and the like, or combinations thereof.

Generally, a threshold value (e.g., threshold score value) refers to a value that is used to separate the positive class from the negative class. The machine learning model as disclosed herein outputs a score (e.g., evaluation score) in between 0 and 1. As would be appreciated by one of skill in the art, and with the help of this disclosure, different thresholds result in different sensitivities, specificities and other performance metrics of the model. A threshold can be chosen to optimize these metrics with a focus on the metrics which are deemed important to a specific task. In some aspects, the threshold value that yields optimal results may be different for different subjects having ASD. In such aspects, a threshold value may be adjusted to a specific value for a specific subject having ASD. A threshold value for a specific subject may also be adjusted over time as the model can get periodically retrained, as more data is acquired and feedback from users is provided, or as additional information (e.g., data) is acquired for that specific subject.

In an aspect, as the machine learning model is running during prospective settings (e.g., the model is deployed), further personalized sensor data are acquired (e.g., collected) from the subjects having ASD. The machine learning model may also be periodically retrained in order to account for data drift and/or concept drift. A data drift can occur when the data distribution of input changes. A concept drift can occur when the functional relationship between the model input values and output changes.

In an aspect, the machine learning model can be customized to a specific subject, wherein the machine learning model may learn patterns of pre-meltdown specific to a subject having ASD. In such aspect, model parameters and/or threshold used to differentiate classes may be adjusted as necessary.

In an aspect, the trained machine learning model can be deployed after development to individuals with ASD or other conditions resulting in meltdowns or outbursts. Referring to the system architecture diagram displayed in FIG. 4, system 400 can be employed for decreasing meltdown incidence and/or severity in a subject having a neurodevelopmental disorder. Sensor data 14 (e.g., motion data, movement data, physiological data, vital signs, sound data, stress data, etc.) can be collected (e.g., acquired) from the individual (e.g., subject 12) wearing the sensors 13 (e.g., wearable sensor, wearable device, etc.). The collected sensor data 14 can be transferred 15 to a smart device 16, wherein the smart device 16 can determine if data manipulation occurs locally (e.g., edge computing) or in the cloud (e.g., cloud computing). In aspects where local or edge compute is utilized, a limited version of filtering may be utilized, for example to limit power and computation use. In aspects where cloud compute is utilized, sensor data can be transmitted to a cloud server for filtering and data processing. Sensor data can be filtered with signal processing as previously described herein, for example by isolating the meltdown-like signal alone and/or filtering other data and background noise. Filtered signals can be further processed to match the data input required for the model, wherein processing can include binning, summarization, aggregation, imputation, and the like, or combinations thereof. Processed data can then be input to the machine learning model 23, wherein the model produces an output (e.g., evaluation score), and wherein the output is compared to a predetermined threshold (e.g., threshold score value). In aspects where the output (e.g., evaluation score) is below (e.g., less than) the threshold (e.g., threshold score value), the data collection, filtering, and processing can repeat in a continuous fashion. In aspects where the output (e.g., evaluation score) is above 31 (e.g., equal to or greater than) the threshold (e.g., threshold score value), the machine learning model 23 can send a signal to a device (e.g., a smart device, such as smart device 16) to trigger the delivery of the audible sound therapy 40 (e.g., a music selection of the user's choice). The sensor data can be substantially continuously monitored, wherein the delivery of the audible sound therapy (e.g., the music) continues until the model output is below the threshold. In some aspects, an alert can be sent (e.g., actively or passively) to a clinician (e.g., a physician or other qualified healthcare professional associated with the subject) in order for the clinician to analyze pre-meltdown severity and/or frequency. The severity of the pre-meltdown can be determined by magnitude of the difference between the evaluation score and the threshold (e.g., level above the threshold). The process can repeat substantially continuously as long as the system is chosen to be active, and the user is utilizing the wearable.

In an aspect, the method for decreasing meltdown incidence and/or severity in a subject having a neurodevelopmental disorder as disclosed herein can advantageously provide a therapeutic intervention (e.g., delivering audible sound therapy) to an individual in a pre-meltdown stage, wherein the meltdowns may be prevented in some instances, or at least have their severity decreased. Additional advantages of the method for decreasing meltdown incidence and/or severity in a subject having a neurodevelopmental disorder as disclosed herein can be apparent to one of skill in the art viewing this disclosure.

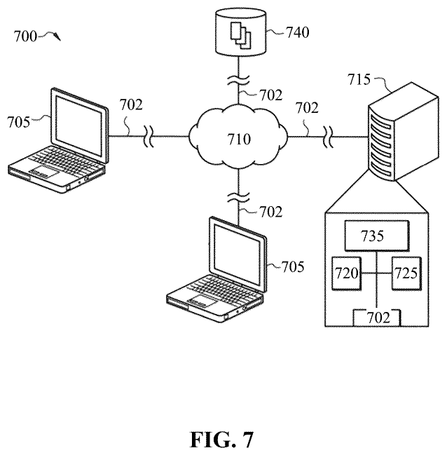

A machine-learning model as disclosed herein is illustrated in the context of FIG. 7. For example, FIG. 7 illustrates an embodiment of a computing system 700 that includes a number of clients 705, a server system 715, and a data repository 740 communicably coupled through a network 710 by one or more communication links 702 (e.g., wireless, wired, or a combination thereof). The computing system 700, generally, can execute applications and analyze data received from sensors, such as may be acquired in the performance of the methods disclosed herein. For instance, the computing system 700 may execute a machine-learning model 735 to as disclosed herein.

In general, the server system 715 can be any server that stores one or more hosted applications, such as, for example, the machine-learning model 735. In some instances, the machine-learning model 735 may be executed via requests and responses sent to users or clients within and communicably coupled to the illustrated computing system 700. In some instances, the server system 715 may store a plurality of various hosted applications, while in other instances, the server system 715 may be a dedicated server meant to store and execute only a single hosted application, such as the machine-learning model 735.

In some instances, the server system 715 may comprise a web server, where the hosted applications represent one or more web-based applications accessed and executed via network 710 by the clients 705 of the system to perform the programmed tasks or operations of the hosted application. At a high level, the server system 715 can comprise an electronic computing device operable to receive, transmit, process, store, or manage data and information associated with the computing system 700. Specifically, the server system 715 illustrated in FIG. 7 can be responsible for receiving application requests from one or more client applications associated with the clients 705 of computing system 700 and responding to the received requests by processing the requests in the associated hosted application and sending the appropriate response from the hosted application back to the requesting client application.

In addition to requests from the clients 705, requests associated with the hosted applications may also be sent from internal users, external or third-party customers, other automated applications, as well as any other appropriate entities, individuals, systems, or computers. As used in the present disclosure and as described in more detail herein, the term “computer” is intended to encompass any suitable processing device. For example, although FIG. 7 illustrates a single server system 715, a computing system 700 can be implemented using two or more server systems 715, as well as computers other than servers, including a server pool. The server system 715 may be any computer or processing device such as, for example, a blade server, general-purpose personal computer (PC), Macintosh, workstation, UNIX-based workstation, or any other suitable device. In other words, the present disclosure contemplates computers other than general-purpose computers, as well as computers without conventional operating systems. Further, the illustrated server system 715 may be adapted to execute any operating system, including Linux, UNIX, Windows, Mac OS, or any other suitable operating system.

In the illustrated embodiment, and as shown in FIG. 7, the server system 715 includes a processor 720, an interface 730, a memory 725, and the machine-learning model 735. The interface 730 is used by the server system 715 for communicating with other systems in a client-server or other distributed environment (including within computing system 700) connected to the network 710 (e.g., clients 705, as well as other systems communicably coupled to the network 710). Generally, the interface 730 comprises logic encoded in software and/or hardware in a suitable combination and operable to communicate with the network 710. More specifically, the interface 730 may comprise software supporting one or more communication protocols associated with communications such that the network 710 or interface's hardware is operable to communicate physical signals within and outside of the illustrated computing system 700.

Although illustrated as a single processor 720 in FIG. 7, two or more processors may be used according to particular needs, desires, or particular embodiments of computing system 700. Each processor 720 may be a central processing unit (CPU), a blade, an application-specific integrated circuit (ASIC), a field-programmable gate array (FPGA), or another suitable component. Generally, the processor 720 executes instructions and manipulates data to perform the operations of server system 715 and, specifically, the machine-learning model 735. Specifically, the server's processor 720 executes the functionality required to receive and respond to requests from the clients 705 and their respective client applications, as well as the functionality required to perform the other operations of the machine-learning model 735.

Regardless of the particular implementation, “software” may include computer-readable instructions, firmware, wired or programmed hardware, or any combination thereof on a tangible medium operable when executed to perform at least the processes and operations described herein. Each software component may be fully or partially written or described in any appropriate computer language including C, C++, C #, Java, Visual Basic, assembler, Perl, any suitable version of 4GL, as well as others. It will be understood that while portions of the software implemented in the context of the embodiments disclosed herein may be shown as individual modules that implement the various features and functionality through various objects, methods, or other processes, the software may instead include a number of sub-modules, third-party services, components, libraries, and such, as appropriate. Conversely, the features and functionality of various components can be combined into single components as appropriate. In the illustrated computing system 700, processor 720 executes one or more hosted applications on the server system 715.

At a high level, the machine-learning model 735 is any application, program, module, process, or other software that may execute, change, delete, generate, or otherwise manage information according to the present disclosure, particularly in response to and in connection with one or more requests received from the illustrated clients 705 and their associated client applications. In certain cases, only one machine-learning model 735 may be located at a particular server system 715. In others, a plurality of related and/or unrelated modeling systems may be stored at a server system 715, or located across a plurality of other server systems 715, as well. In certain cases, computing system 700 may implement a composite hosted application. For example, portions of the composite application may be implemented as Enterprise Java Beans (EJBs) or design-time components may have the ability to generate run-time implementations into different platforms, such as J2EE (Java 2 Platform, Enterprise Edition), ABAP (Advanced Business Application Programming) objects, or Microsoft's .NET, among others. Additionally, the hosted applications may represent web-based applications accessed and executed by clients 705 or client applications via the network 710 (e.g., through the Internet).

Further, while illustrated as internal to server system 715, one or more processes associated with machine-learning model 735 may be stored, referenced, or executed remotely. For example, a portion of the machine-learning model 735 may be a web service associated with the application that is remotely called, while another portion of the machine-learning model 735 may be an interface object or agent bundled for processing at a client 705 located remotely. Moreover, any or all of the machine-learning model 735 may be a child or sub-module of another software module or enterprise application (not illustrated) without departing from the scope of this disclosure. Still further, portions of the machine-learning model 735 may be executed by a user working directly at server system 715, as well as remotely at clients 705.

The server system 715 also includes memory 725. Memory 725 may include any memory or database module and may take the form of volatile or non-volatile memory. The illustrated computing system 700 of FIG. 2 also includes one or more clients 705. Each client 705 may be any computing device operable to connect to or communicate with at least the server system 715 and/or via the network 710 using a wireline or wireless connection.

The illustrated data repository 740 may be any database or data store operable to store data, such as data received from a sensor. Generally, the data may comprise inputs to the machine-learning model 735, historical information, operational information, and/or output data from the machine-learning model 735.

The functionality of one or more of the components disclosed with respect to FIG. 7, such as the server system 715 or the clients 705, can be carried out on a computer or other device comprising a processor (e.g., a desktop computer, a laptop computer, a tablet, a server, a smartphone, smartwatch, or some combination thereof). Generally, such a computer or other computing device may include a processor (which may be referred to as a central processor unit or CPU) that is in communication with memory devices including secondary storage, read-only memory (ROM), random access memory (RAM), input/output (I/O) devices, and network connectivity devices. The processor may be implemented as one or more CPU chips.

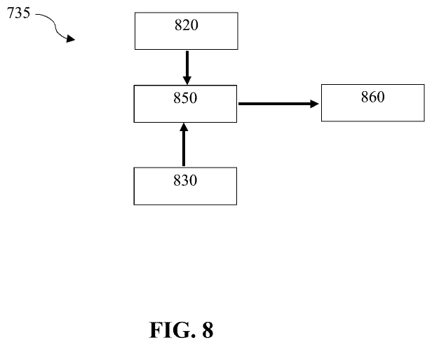

FIG. 8 depicts an example of the operation of the machine-learning model 735 of FIG. 7. In the embodiment of FIG. 8, the machine-learning model 735 comprises a machine-learning module 850 coupled to one or more data stores, for example, data within the data repository 740. For instance, in the embodiment of FIG. 8, the data within the data repository 740 of FIG. 7 may include data from a training data store 820 and/or inputs 830.

As also shown in FIG. 8, the machine-learning module 850 can access data, such as data from the training data store 820, receive inputs 830, and provide an output 860 based upon the inputs 830 and data retrieved from the training data store 820. Generally, the machine-learning module 850 utilizes data stored in the training data store 820, for example, sensor data as disclosed herein, to enable the machine-learning module 850 to predictively determine the state of a subject based upon additional sensor data evaluated by the machine-learning module 850.

In some embodiments, at least a portion of the data stored in the training data store 820 may be characterized as “training data” that is used to train the machine-learning module 850. As will be appreciated by the ordinarily-skilled artisan upon viewing the instant disclosure, although the Figures illustrate an aspect in which the training data are stored in a single “store” (e.g., at least a portion of the training data store 820), additionally or alternatively, in some embodiments the training data may be stored in multiple stores in one or more locations. Additionally, in some embodiments, the training data (e.g., at least a portion of the data stored in the training data store 820) may be subdivided into two or more subgroups, for example, a training data subset, one or more evaluation and/or testing data subsets, or combinations thereof.

### Additional Embodiments

A 1^{st} embodiment is a method comprising (a) acquiring sensor data from one or more wearable sensors configured to be worn by a subject, wherein the sensor data comprise motion data, sound data, physiological data, or combinations thereof of the subject; (b) comparing the sensor data of the subject with target data, wherein the motion data, the sound data, the physiological data, or combinations thereof of the subject are compared with target motion data, target sound data, target physiological data, or combinations thereof, respectively; (c) determining, in any sequence, at least one of the following: that the motion data of the subject is equal to or exceed the target motion data; that the sound data of the subject is equal to or exceed the target sound data; and that the physiological data of the subject is equal to or exceed the target physiological data; and (d) responsive to step (c), delivering audible sound therapy to the subject, wherein the audible sound therapy comprises an audio sound track, wherein the audio sound track is characterized by a track rhythm and by a track beat, wherein the audio sound track is familiar to the subject, and wherein the audio sound track is repeated at least until it is determined, in any sequence, at least one of the following: that the target motion data exceed the motion data of the subject; that the target sound data exceed the sound data of the subject; and that the target physiological data exceed the physiological data of the subject.

A 2^{nd} embodiment is the method of the 1^{st} embodiment, wherein the subject has a neurodevelopmental disorder comprising autism spectrum disorder (ASD), sensory processing disorder, or combinations thereof.

A 3^{rd} embodiment is the method of one of the 1^{st} and the 2^{nd} embodiments, wherein the subject has ASD, and wherein the sensor data being equal to or exceeding target data correlates with the onset of a pre-meltdown stage for the subject.

A 4^{th} embodiment is the method of the 3^{rd} embodiment, wherein the step of delivering audible sound therapy to the subject prevents the onset of a meltdown stage for the subject or decreases the severity of a meltdown stage for the subject.

A 5^{th} embodiment is the method of one of the 1^{st} through the 4^{th} embodiments, wherein the audio sound track comprises a song, a music album, an audio book chapter, an audio book, a recited poem, a collection of recited poems, or combinations thereof.

A 6^{th} embodiment is the method of one of the 1^{st} through the 5^{th} embodiments, wherein the motion data comprise motion frequency and/or motion intensity; wherein the target motion data comprise target motion frequency and/or target motion intensity, respectively; and wherein the audible sound therapy is delivered to the subject at least until the target motion frequency and/or target motion intensity exceed the motion frequency and/or motion intensity, respectively, of the subject.

A 7^{th} embodiment is the method of the 6^{th} embodiment, wherein the track rhythm and/or the track beat corresponds to a motion frequency of the subject that is less than the target motion frequency.

An 8^{th} embodiment is the method of the 6^{th} embodiment, wherein the subject achieves a motion frequency that is less than the target motion frequency as a result of the audible sound therapy.

A 9^{th} embodiment is the method of the 6^{th} embodiment, wherein the track rhythm and/or the track beat provide for the subject achieving a rhythmic motion that is substantially synchronized to the track rhythm and/or to the track beat, and wherein the target motion frequency exceeds the motion frequency of the subject.

A 10^{th} embodiment is the method of the 9^{th} embodiment, wherein the rhythmic motion comprises a body motion, a torso motion, a limb motion, an arm motion, a hand motion, a finger motion, a leg motion, a foot motion, a toe motion, a knee motion, a head motion, or combinations thereof.

An 11^{th} embodiment is the method of one of the P t through the 10^{th} embodiments, wherein the physiological data comprise heart rate, blood pressure, respiration rate, breathing pattern, oxygen saturation rate, muscle tension level, temperature, one or more electrocardiogram (ECG) features, one of more electromyogram (EMG) features, or combinations thereof; and wherein the audible sound therapy is delivered to the subject at least until the target physiological data exceed the physiological data of the subject.

A 12^{th} embodiment is the method of one of the P t through the 11^{th} embodiments, wherein the sound data comprise vocal sounds produced by the subject; and wherein the audible sound therapy is delivered to the subject at least until the target sound data exceed the sound data of the subject.

A 13^{th} embodiment is the method of one of the P t through the 12^{th} embodiments, wherein the one or more wearable sensors comprise at least one sensor configured to detect motion data, at least one sensor configured to detect sound data, at least one sensor configured to detect physiological data, or combinations thereof.

A 14^{th} embodiment is the method of one of the P t through the 13^{th} embodiments, wherein at least one of the one or more wearable sensors is wireless.

A 15^{th} embodiment is the method of one of the P t through the 13^{th} embodiments, wherein at least one of the one or more wearable sensors is wired.

A 16^{th} embodiment is the method of one of the P t through the 15^{th} embodiments, wherein the audible sound therapy is delivered via speakers, earbuds, or headphones.

A 17^{th} embodiment is the method of one of the P t through the 16^{th} embodiments excluding an input from a caregiver for delivering the audible sound therapy to the subject.

An 18^{th} embodiment is the method of one of the 1^{st} through the 17^{th} embodiments further comprising receiving, by a control system, the sensor data from the one or more wearable sensors; wherein the control system comprises at least one processor and at least one controller; wherein the at least one processor compares the sensor data to the target data; wherein, when at least one of the sensor data are equal to or exceed the target data, the at least one processor signals the at least one controller; and wherein the at least one controller delivers the audible sound therapy to the subject.

A 19^{th} embodiment is the method of the 18^{th} embodiment, wherein the control system provides for real-time delivery of the audible sound therapy to the subject.

A 20^{th} embodiment is the method of the 18^{th} embodiment, wherein the control system selects the audio sound track that is familiar to the subject from a library of audio sound tracks that are familiar to the subject; and wherein said selection is based on the type of sensor data of the subject that is equal to or exceed the target data and/or the magnitude of difference between the sensor data of the subject and the target data.

An 21^{st} embodiment is the method of one of the 1^{st} through the 17^{th} embodiments, wherein step (a) of acquiring sensor data from one or more wearable sensors further comprises receiving, by at least one computing device, the sensor data from the one or more wearable sensors.

A 22^{nd} embodiment is the method of the 21^{st} embodiment further comprising filtering, by the at least one computing device, the sensor data from the one or more wearable sensors to yield filtered sensor data.

A 23^{rd} embodiment is the method of the 22^{nd} embodiment, wherein filtering the sensor data comprises removing noise from the sensor data, isolating a signal associated with a specific subject behavior, identifying a signal associated with a specific subject behavior, or combinations thereof; and wherein the specific subject behavior is voluntary or involuntary.

A 24^{th} embodiment is the method of the 23^{rd} embodiment, wherein the specific subject behavior comprises a pre-meltdown behavior.

A 25^{th} embodiment is the method of one of the 21^{st} through the 24^{th} embodiments further comprising processing, by the at least one computing device, the sensor data and/or the filtered sensor data to yield processed sensor data.

A 26^{th} embodiment is the method of one of the 21^{st} through the 25^{th} embodiments, wherein the sensor data are filtered and/or processed via edge computing, via cloud computing, or via both edge computing and cloud computing.

A 27^{th} embodiment is the method of one of the 25^{th} and the 26^{th} embodiments, wherein the sensor data and/or the filtered sensor data are time-series data, and wherein processing the sensor data comprise converting, by the at least one computing device, the time-series data to tabular data.

A 28^{th} embodiment is the method of one of the 21^{st} through the 27^{th} embodiments, wherein step (b) of comparing the sensor data of the subject with target data further comprises providing, by the at least one computing device, an input to an algorithm based on at least one machine learning model that is trained with sensor data from the subject and/or with sensor data from at least one additional subject, wherein the algorithm is configured to evaluate the sensor data of the subject with respect to the target data.

A 29^{th} embodiment is the method of the 28^{th} embodiment, wherein the at least one machine learning model is further trained with sensor data from the subject only.

A 30^{th} embodiment is the method of one of the 28^{th} and the 29^{th} embodiments, wherein the at least one machine learning model further excludes training with data from a different subject.

A 31^{st} embodiment is the method of one of the 28^{th} through the 30^{th} embodiments, wherein the input to the algorithm comprises tabular data.

A 32^{nd} embodiment is the method of one of the 28^{th} through the 31^{st} embodiments, wherein the algorithm is generated using a machine learning technique selected from the group consisting of a deep learning model, a recurrent neural networks model, a classical tree-based machine learning model, a decision tree type model, a regression type model, and combinations thereof.

A 33^{rd} embodiment is the method of one of the 28^{th} through the 32^{nd} embodiments, wherein the algorithm is deployed via edge computing, via cloud computing, or via both edge computing and cloud computing.

A 34^{th} embodiment is the method of one of the 21^{st} through the 33^{rd} embodiments, wherein step (c) of determining further comprises determining, by the at least one computing device, that the sensor data of the subject is equal to or exceed the target data.

A 35^{th} embodiment is the method of the 34^{th} embodiment, wherein determining, by the at least one computing device, that the sensor data of the subject is equal to or exceed the target data further comprises comparing at least one evaluation score with at least one threshold score value and determining that the evaluation score is equal to or exceeds the threshold score value.

A 36^{th} embodiment is the method of one of the 21^{st} through the 35^{th} embodiments, wherein step (d) of delivering audible sound therapy to the subject further comprises delivering audible sound therapy to the subject by the at least one computing device at least until the threshold score value exceeds the evaluation score.

A 37^{th} embodiment is the method of one of the 1^{st} through the 36^{th} embodiments further comprising informing the subject and/or a caregiver that the sensor data of the subject exceed the target data.

A 38^{th} embodiment is a method comprising (a) acquiring sensor data from one or more wearable sensors configured to be worn by a subject, wherein the sensor data comprise motion data, sound data, physiological data, or combinations thereof of the subject; (b) transmitting the sensor data from the one or more wearable sensors to at least one computing device; (c) optionally filtering, by the at least one computing device, the sensor data to yield filtered sensor data; (d) processing, by the at least one computing device, the sensor data and/or the filtered sensor data to yield processed sensor data; (e) evaluating the sensor data of the subject with respect to target data, wherein the motion data, the sound data, the physiological data, or combinations thereof of the subject are evaluated with respect to target motion data, target sound data, target physiological data, or combinations thereof, respectively; and wherein evaluating the sensor data of the subject with respect to the target data comprises providing, by the at least one computing device, an input to an algorithm based on at least one machine learning model that is trained with sensor data, filtered sensor data, processed sensor data, or combinations thereof from the subject and/or with data from at least one additional subject, wherein the algorithm is configured to evaluate the sensor data of the subject with respect to the target data, provide an evaluation score, and compare the evaluation score with a threshold score value; (f) determining, by the at least one computing device, in any sequence, at least one of the following: that the motion data of the subject is equal to or exceed the target motion data; that the sound data of the subject is equal to or exceed the target sound data; and that the physiological data of the subject is equal to or exceed the target physiological data; wherein the sensor data of the subject being equal to or exceeding the target data corresponds to the evaluation score being equal to or exceeding a threshold score value; and (g) responsive to step (f), delivering, by the at least one computing device, audible sound therapy to the subject, wherein the audible sound therapy comprises an audio sound track, wherein the audio sound track is characterized by a track rhythm and by a track beat, wherein the audio sound track is familiar to the subject, and wherein the audio sound track is repeated at least until it is determined, in any sequence, at least one of the following: that the target motion data exceed the motion data of the subject; that the target sound data exceed the sound data of the subject; and that the target physiological data exceed the physiological data of the subject; wherein the target data exceeding the sensor data of the subject corresponds to the threshold score value being equal to or exceeding the evaluation score.

A 39^{th} embodiment is the method of the 38^{th} embodiment, wherein the subject has autism spectrum disorder (ASD); wherein the evaluation score being equal to or exceeding a threshold score value correlates with the onset of a pre-meltdown stage for the subject; and wherein the step of delivering audible sound therapy to the subject prevents the onset of a meltdown stage for the subject or decreases the severity of a meltdown stage for the subject.

A 40^{th} embodiment is the method of one of the 38^{th} and the 39^{th} embodiments, wherein the audio sound track comprises a song, a music album, an audio book chapter, an audio book, a recited poem, a collection of recited poems, or combinations thereof.

A 41^{st} embodiment is the method of one of the 38^{th} through the 40^{th} embodiments, wherein the sensor data are selected from the group consisting of motion data, motion frequency, motion intensity, motion pattern, physiological data, heart rate, blood pressure, respiration rate, breathing pattern, oxygen saturation rate, muscle tension level, temperature, one or more electrocardiogram (ECG) features, one or more electromyogram (EMG) features, sound data, vocal sounds produced by the subject, and combinations thereof.

A 42nd embodiment is the method of one of the 38^{th} through the 41^{st} embodiments, wherein data are filtered and or processed, by the at least one computing device, via edge computing, via cloud computing, or via both edge computing and cloud computing.

A 43^{rd} embodiment is the method of one of the 38^{th} through the 42^{nd} embodiments, wherein step (e) of evaluating the sensor data of the subject with respect to target data comprises deploying the algorithm via edge computing, via cloud computing, or via both edge computing and cloud computing.

A 44^{th} embodiment is a method comprising (a) acquiring sensor data from one or more wearable sensors configured to be worn by a subject, wherein the sensor data comprise motion data, sound data, physiological data, or combinations thereof of the subject, wherein the subject has autism spectrum disorder (ASD), and wherein the sensor data are time-series data; (b) transmitting the sensor data from the one or more wearable sensors to at least one computing device; (c) filtering, by the at least one computing device, the sensor data to yield filtered sensor data; (d) processing, by the at least one computing device, the filtered sensor data to yield processed sensor data, wherein the processed sensor data are tabular data; (e) evaluating the sensor data of the subject with respect to target data, wherein the motion data, the sound data, the physiological data, or combinations thereof of the subject are evaluated with respect to target motion data, target sound data, target physiological data, or combinations thereof, respectively; wherein evaluating the sensor data of the subject with respect to the target data comprises providing, by the at least one computing device, an input to an algorithm based on at least one machine learning model that is trained with sensor data, filtered sensor data, processed sensor data, or combinations thereof from the subject and/or with data from at least one additional subject; and wherein the algorithm is configured to evaluate the sensor data of the subject with respect to the target data, provide an evaluation score, and compare the evaluation score with a threshold score value; and (f) determining, by the at least one computing device, that the evaluation score is equal to or exceeds a threshold score value; wherein the evaluation score being equal to or exceeding a threshold score value corresponds to at least one of the following: the motion data of the subject being equal to or exceeding the target motion data; the sound data of the subject being equal to or exceeding the target sound data; and the physiological data of the subject being equal to or exceeding the target physiological data; and wherein the evaluation score being equal to or exceeding a threshold score value correlates with the onset of a pre-meltdown stage for the subject.

A 45^{th} embodiment is the method of the 44^{th} embodiment further comprising, responsive to step (f), delivering, by the at least one computing device, audible sound therapy to the subject; wherein the audible sound therapy comprises an audio sound track; wherein the audio sound track is characterized by a track rhythm and by a track beat; wherein the audio sound track is familiar to the subject; wherein the audio sound track is repeated at least until it is determined, by the at least one computing device, that the threshold score value is equal to or exceeds an evaluation score; and wherein delivering the audible sound therapy to the subject prevents the onset of a meltdown stage for the subject or decreases the severity of a meltdown stage for the subject.

A 46^{th} embodiment is the method of the 45^{th} embodiment further comprising informing the subject and/or a caregiver of the onset of the pre-meltdown stage.

A 47^{th} embodiment is the method of one of the 44^{th} through the 46^{th} embodiments, wherein the sensor data are selected from the group consisting of motion data, motion frequency, motion intensity, motion pattern, physiological data, heart rate, blood pressure, respiration rate, breathing pattern, oxygen saturation rate, muscle tension level, temperature, one or more electrocardiogram (ECG) features, one of more electromyogram (EMG) features, sound data, vocal sounds produced by the subject, and combinations thereof.

A 48^{th} embodiment is the method of one of the 44^{th} through the 47^{th} embodiments, wherein the sensor data are filtered and/or processed via edge computing, via cloud computing, or via both edge computing and cloud computing.

A 49^{th} embodiment is the method of one of the 44^{th} through the 48^{th} embodiments, wherein step (e) of evaluating the sensor data of the subject with respect to target data comprises deploying the algorithm via edge computing, via cloud computing, or via both edge computing and cloud computing.

A 50^{th} embodiment is a method comprising (a) acquiring sensor data from one or more wearable sensors configured to be worn by a subject, wherein the subject has autism spectrum disorder (ASD), and wherein the sensor data are time-series data; (b) transmitting the sensor data from the one or more wearable sensors to at least one computing device; (c) filtering, by the at least one computing device, the sensor data to yield filtered sensor data; (d) processing, by the at least one computing device, the filtered sensor data to yield processed sensor data, wherein the processed sensor data are tabular data; (e) evaluating the sensor data of the subject with respect to target data; wherein evaluating the sensor data of the subject with respect to the target data comprises providing, by the at least one computing device, an input to an algorithm based on at least one machine learning model that is trained with sensor data, filtered sensor data, processed sensor data, or combinations thereof from the subject and/or with data from at least one additional subject, wherein the algorithm is configured to evaluate the sensor data of the subject with respect to the target data, provide an evaluation score, and compare the prediction score with a threshold score value; (f) determining, by the at least one computing device, that the evaluation score is equal to or exceeds a threshold score value, wherein the evaluation score being equal to or exceeding a threshold score value corresponds to the sensor data of the subject being equal to or exceeding the target data, and wherein the sensor data being equal to or exceeding target data correlates with the onset of a pre-meltdown stage for the subject; and (g) responsive to step (f), delivering, by the at least one computing device, audible sound therapy to the subject, wherein the audible sound therapy comprises an audio sound track, wherein the audio sound track is characterized by a track rhythm and by a track beat, wherein the audio sound track is familiar to the subject, and wherein the audio sound track is repeated at least until it is determined, by the at least one computing device, that the threshold score value is equal to or exceeds an evaluation score; and wherein delivering the audible sound therapy to the subject prevents the onset of a meltdown stage for the subject or decreases the severity of a meltdown stage for the subject.

A 51^{st} embodiment is a system comprising one or more wearable sensors configured to detect sensor data of a subject; wherein the one or more wearable sensors comprise at least one sensor configured to detect motion data, at least one sensor configured to detect sound data, at least one sensor configured to detect physiological data, or combinations thereof; and a control system configured to receive the sensor data of the subject from the one or more wearable sensors; wherein the control system comprises at least one processor and at least one controller; wherein the at least one processor compares the sensor data to target data; wherein, when at least one of the sensor data are equal to or exceed the target data, the at least one processor is configured to signal the at least one controller; and wherein the at least one controller delivers an audible sound therapy to the subject; wherein the audible sound therapy comprises an audio sound track, wherein the audio sound track is characterized by a track rhythm and by a track beat, wherein the audio sound track is familiar to the subject, and wherein the audio sound track is repeated at least until it is determined that the target data exceed the sensor data of the subject.

A 52^{nd} embodiment is the system of the 51^{st} embodiment, wherein the at least one processor compares the motion data, the sound data, the physiological data, or combinations thereof of the subject with target motion data, target sound data, target physiological data, or combinations thereof, respectively.

A 53^{rd} embodiment is the system of one of the 51^{st} and the 52^{nd} embodiments, wherein the control system further comprises (i) an algorithm based on at least one machine learning model that is trained with sensor data from the subject and/or with data from at least one additional subject, wherein the algorithm is configured to evaluate the sensor data of the subject with respect to target data; and (ii) a non-transitory computer readable medium that stores instructions that when executed by the processor, causes the processor to: receive, using the control system, an input comprising sensor data of the subject; provide, by the control system, the input to the algorithm; determine, by the control system, an evaluation result comprising an indication that the sensor data of the subject is equal to or exceed the target data by using the algorithm; wherein the indication that the sensor data of the subject is equal to or exceed the target data is an evaluation score being equal to or greater than a threshold score value; and deliver, by the control system, audible sound therapy to the subject, wherein the audible sound therapy comprises an audio sound track, wherein the audio sound track is familiar to the subject, and wherein the audio sound track is configured to be repeated at least until the target data exceed the sensor data of the subject.

A 54^{th} embodiment is the system of one of the 51^{st} through the 53^{rd} embodiments, wherein the sensor data comprise data obtained without requiring assistance from a caregiver.

A 55^{th} embodiment is the system of one of the 51^{st} through the 54^{th} embodiments, further comprising an audio speaker, earbuds, and/or headphones, wherein the audible sound therapy is delivered via the audio speaker, earbuds, and/or headphones without requiring assistance from a caregiver.

A 56^{th} embodiment is the system of one of the 51^{st} through the 55^{th} embodiments, wherein the subject has autism spectrum disorder (ASD); wherein the sensor data being equal to or exceeding target data correlates with the onset of a pre-meltdown stage for the subject; and wherein the audible sound therapy prevents the onset of a meltdown stage for the subject or decreases the severity of a meltdown stage for the subject.

A 57^{th} embodiment is a system comprising one or more wearable sensors configured to detect sensor data of a subject; wherein the one or more wearable sensors comprise at least one sensor configured to detect motion data, at least one sensor configured to detect sound data, at least one sensor configured to detect physiological data, or combinations thereof; and a computing system configured to receive the sensor data of the subject from the one or more wearable sensors; wherein the computing system comprises (i) at least one processor; (ii) an algorithm based on at least one machine learning model that is trained with sensor data from the subject and/or with data from at least one additional subject, wherein the algorithm is configured to evaluate the sensor data of the subject with respect to target data, provide an evaluation score, and compare the evaluation score with a threshold score value; and (iii) a non-transitory computer readable medium that stores instructions that when executed by the processor, causes the processor to: receive, using the computing system, an input comprising sensor data of the subject; provide, by the computing system, the input to the algorithm; and determine, by the computing system, an evaluation result comprising an indication that the sensor data of the subject is equal to or exceed the target data by using the algorithm; wherein the indication that the sensor data of the subject is equal to or exceed the target data is the evaluation score being equal to or greater than the threshold score value; and wherein the sensor data being equal to or exceeding target data correlates with the onset of a pre-meltdown stage for the subject.

A 58^{th} embodiment is the system of the 57^{th} embodiment, wherein the processor is configured to deliver, by the computing system, an audible sound therapy to the subject; wherein the audible sound therapy comprises an audio sound track, wherein the audio sound track is characterized by a track rhythm and by a track beat, wherein the audio sound track is familiar to the subject, and wherein the audio sound track is repeated at least until it is determined, by the computing system, that the threshold score value exceeds the evaluation score.

A 59^{th} embodiment is the system of one of the 57^{th} and the 58^{th} embodiments, wherein the subject has autism spectrum disorder (ASD); and wherein the audible sound therapy prevents the onset of a meltdown stage for the subject or decreases the severity of a meltdown stage for the subject.

A 60^{th} embodiment is the system of one of the 57^{th} through the 59^{th} embodiments, wherein the computing system is further configured to notify the subject and/or a caregiver of the onset of a pre-meltdown stage for the subject.

A 61^{st} embodiment is the system of one of the 57^{th} through the 60^{th} embodiments, wherein the sensor data are time-series data; wherein the computing system is configured to filter the sensor data to yield filtered sensor data; wherein the computing system is configured to process the filtered sensor data to yield processed sensor data, wherein the processed sensor data are tabular data; and wherein the input to the algorithm comprises tabular data.

A 62^{nd} embodiment is a method comprising receiving sensor data from one or more wearable sensors configured to be worn by a subject, wherein the sensor data comprise motion data, sound data, physiological data, or combinations thereof of the subject; determining that the sensor data deviates from an ordinary profile associated with the subject; and responsive to the determination that the sensor data deviates from the ordinary profile associated with the subject, delivering audible sound therapy to the subject, wherein the audible sound therapy comprises an audio sound track associated with the subject.

A 63^{rd} embodiment is the method of the 62^{nd} embodiment, wherein the subject has a neurodevelopmental disorder comprising autism spectrum disorder (ASD), sensory processing disorder, or combinations thereof.

A 64^{th} embodiment is the method of one of the 62^{nd} and the 63^{rd} embodiments, wherein the subject has ASD, and wherein determination that the sensor data deviates from the ordinary profile associated with the subject correlates with the onset of a pre-meltdown stage for the subject.

A 65^{th} embodiment is the method of the 64^{th} embodiment, wherein the step of delivering audible sound therapy to the subject prevents the onset of a meltdown stage for the subject or decreases the severity of a meltdown stage for the subject.

A 66^{th} embodiment is the method of one of the 62^{nd} through the 65^{th} embodiments, wherein the audio sound track comprises a song, a music album, an audio book chapter, an audio book, a recited poem, a collection of recited poems, or combinations thereof.

A 67^{th} embodiment is the method of one of the 62^{nd} through the 66^{th} embodiments, wherein the motion data comprise motion frequency and/or motion intensity; wherein the target motion data comprise target motion frequency and/or target motion intensity, respectively; and wherein the audible sound therapy is delivered to the subject at least until the target motion frequency and/or target motion intensity exceed the motion frequency and/or motion intensity, respectively, of the subject.

A 68^{th} embodiment is the method of the 67^{th} embodiment, wherein the audio sound track is characterized by a track rhythm and by a track beat, wherein the track rhythm and/or the track beat corresponds to a motion frequency of the subject that is less than the target motion frequency.

A 69^{th} embodiment is the method of the 67^{th} embodiment, wherein the subject achieves a motion frequency that is less than the target motion frequency as a result of the audible sound therapy.

A 70^{th} embodiment is the method of the 67^{th} embodiment, wherein the track rhythm and/or the track beat provide for the subject achieving a rhythmic motion that is substantially synchronized to the track rhythm and/or to the track beat, and wherein the target motion frequency exceeds the motion frequency of the subject.

A 71^{st} embodiment is the method of the 70^{th} embodiment, wherein the rhythmic motion comprises a body motion, a torso motion, a limb motion, an arm motion, a hand motion, a finger motion, a leg motion, a foot motion, a toe motion, a knee motion, a head motion, or combinations thereof.

A 72^{nd} embodiment is the method of one of the 62^{nd} through the 71^{st} embodiments, wherein the physiological data comprise heart rate, blood pressure, respiration rate, breathing pattern, oxygen saturation rate, muscle tension level, temperature, one or more electrocardiogram (ECG) features, one of more electromyogram (EMG) features, or combinations thereof; and wherein the audible sound therapy is delivered to the subject at least until the target physiological data exceed the physiological data of the subject.

A 73^{rd} embodiment is the method of one of the 62^{nd} through the 72^{nd} embodiments, wherein the sound data comprise vocal sounds produced by the subject; and wherein the audible sound therapy is delivered to the subject at least until the target sound data exceed the sound data of the subject.

A 74^{th} embodiment is the method of one of the 62^{nd} through the 73^{rd} embodiments, wherein the one or more wearable sensors comprise at least one sensor configured to detect motion data, at least one sensor configured to detect sound data, at least one sensor configured to detect physiological data, or combinations thereof.

A 75^{th} embodiment is the method of one of the 62^{nd} through the 74^{th} embodiments, wherein at least one of the one or more wearable sensors is wireless.

A 76^{th} embodiment is the method of one of the 62^{nd} through the 74^{th} embodiments, wherein at least one of the one or more wearable sensors is wired.

A 77^{th} embodiment is the method of one of the 62^{nd} through the 76^{th} embodiments, wherein the audible sound therapy is delivered via speakers, earbuds, or headphones.

A 78^{th} embodiment is the method of one of the 62^{nd} through the 77^{th} embodiments excluding an input from a caregiver for delivering the audible sound therapy to the subject.

A 79^{th} embodiment is the method of one of the 62^{nd} through the 78^{th} embodiments further comprising receiving, by a control system, the sensor data from the one or more wearable sensors; wherein the control system comprises at least one processor and at least one controller; wherein the at least one processor compares the sensor data to the ordinary profile; wherein, when the sensor data deviates from the ordinary profile, the at least one processor signals the at least one controller and the at least one controller delivers the audible sound therapy to the subject.

An 80^{th} embodiment is the method of the 79^{th} embodiment, wherein the control system provides for real-time delivery of the audible sound therapy to the subject.

An 81^{st} embodiment is the method of the 79^{th} embodiment, wherein the control system selects the audio sound track associated with the subject from a library of audio sound tracks that are associated with the subject; and wherein the selected audio sound track is based on the type of sensor data that deviates from the ordinary profile.

An 82^{nd} embodiment is the method of one of the 62^{nd} through the 78^{th} embodiments, wherein step of receiving sensor data from one or more wearable sensors comprises receiving, by at least one computing device, the sensor data from the one or more wearable sensors.

An 83^{rd} embodiment is the method of the 82^{nd} embodiment further comprising filtering, by the at least one computing device, the sensor data from the one or more wearable sensors to yield filtered sensor data.

An 84^{th} embodiment is the method of the 83^{rd} embodiment, wherein filtering the sensor data comprises removing noise from the sensor data, isolating a signal associated with a specific subject behavior, identifying a signal associated with a specific subject behavior, or combinations thereof; and wherein the specific subject behavior is voluntary or involuntary.

An 85^{th} embodiment is the method of the 84^{th} embodiment, wherein the specific subject behavior comprises a pre-meltdown behavior.

An 86^{th} embodiment is the method of one of the 82^{nd} through the 85^{th} embodiments further comprising processing, by the at least one computing device, the sensor data and/or the filtered sensor data to yield processed sensor data.

An 87^{th} embodiment is the method of one of the 82^{nd} through the 86^{th} embodiments, wherein the sensor data are filtered and/or processed via edge computing, via cloud computing, or via both edge computing and cloud computing.

An 88^{th} embodiment is the method of one of the 86^{th} and the 87^{th} embodiments, wherein the sensor data and/or the filtered sensor data are time-series data, and wherein processing the sensor data comprise converting, by the at least one computing device, the time-series data to tabular data.

An 89^{th} embodiment is the method of one of the 82^{nd} through the 88^{th} embodiments, wherein the step determining that the sensor data deviates from the ordinary profile associated with the subject comprises providing, by the at least one computing device, an input to an algorithm based on at least one machine learning model that is trained with sensor data from the subject and/or with sensor data from at least one additional subject, wherein the algorithm is configured to evaluate the sensor data of the subject with respect to the normal profile.

A 90^{th} embodiment is the method of the 89^{th} embodiment, wherein the at least one machine learning model is further trained with sensor data from the subject only.

A 91^{st} embodiment is the method of one of the 89^{th} and the 90^{th} embodiments, wherein the at least one machine learning model further excludes training with data from a different subject.

A 92^{nd} embodiment is the method of one of the 89^{th} through the 91^{st} embodiments, wherein the input to the algorithm comprises tabular data.

A 93^{rd} embodiment is the method of one of the 89^{th} through the 92^{nd} embodiments, wherein the algorithm is generated using a machine learning technique selected from the group consisting of a deep learning model, a recurrent neural networks model, a classical tree-based machine learning model, a decision tree type model, a regression type model, and combinations thereof.

A 94^{th} embodiment is the method of one of the 89^{th} through the 93^{rd} embodiments, wherein the algorithm is deployed via edge computing, via cloud computing, or via both edge computing and cloud computing.

A 95^{th} embodiment is the method of one of the 82^{nd} through the 94^{th} embodiments, wherein step of determining that the sensor data deviates from the ordinary profile associated with the subject comprises a determination by the at least one computing device.

A 96^{th} embodiment is the method of the 94^{th} embodiment, wherein determining, by the at least one computing device, that the sensor data deviates from the ordinary profile associated with the subject further comprises comparing at least one evaluation score with at least one threshold score value and determining that the evaluation score is equal to or exceeds the threshold score value.

A 97^{th} embodiment is the method of one of the 82^{nd} through the 96^{th} embodiments, wherein the step of delivering audible sound therapy to the subject further comprises delivering audible sound therapy to the subject by the at least one computing device at least until the threshold score value does not exceed the evaluation score.

A 98^{th} embodiment is the method of one of the 62^{nd} through the 97^{th} embodiments further comprising informing the subject and/or a caregiver that the sensor data of the subject exceed the target data.

A 99^{th} embodiment is a method comprising receiving training sensor data from one or more wearable sensors configured to be worn by a subject, wherein the training sensor data comprises motion data associated with the subject, sound data associated with the subject, physiological data associated with the subject, or combinations thereof, wherein the training sensor data is categorized as an ordinary state, a pre-meltdown state, a meltdown state, or combinations thereof; and processing the training sensor data to yield an algorithm that is configured to evaluate sensor data associated with the subject to determine whether the evaluated sensor data is indicative of the ordinary state, the pre-meltdown stage, or the meltdown state of the subject.

An 100^{th} embodiment is the method of the 99^{th} embodiment, wherein the training sensor data is processed via at least one machine learning model.

An 101^{st} embodiment is the method of the 100^{th} embodiment, wherein the at least one machine learning model is further trained with sensor data from the subject only.

An 102^{nd} embodiment is the method of the 100^{th} embodiment, wherein the at least one machine learning model is further trained with data from a different subject.

An 103^{rd} embodiment is the method of one of the 99^{th} through the 102^{nd} embodiments, wherein the input to the algorithm comprises tabular data.

An 104^{th} embodiment is the method of one of the 99^{th} through the 103^{rd} embodiments, wherein the prediction algorithm is generated using a machine learning technique selected from the group consisting of a deep learning model, a recurrent neural networks model, a classical tree-based machine learning model, a decision tree type model, a regression type model, and combinations thereof.

An 105^{th} embodiment is the method of one of the 89^{th} through the 93^{rd} embodiments, wherein the algorithm is deployed via edge computing, via cloud computing, or via both edge computing and cloud computing.

An 106^{th} embodiment is the method of the 94^{th} embodiment, wherein processing the training sensor data to yield the algorithm yields an ordinary profile associated with the subject, at least one threshold score value associated with the subject, or both.

An 107^{th} embodiment is a method comprising delivering audible sound therapy to a subject, wherein the audible sound therapy comprises an audio sound track; and receiving sensor data from one or more wearable sensors worn by the subject, wherein the sensor data comprise motion data, sound data, physiological data, or combinations thereof of the subject that is indicative of an activity of the subject during delivery of the audible sound therapy to the subject; determining that the audio sound track is effective to prevent onset of a meltdown stage for the subject or decrease severity of a meltdown stage for the subject; and associating the audio sound track with the subject.

An 108^{th} embodiment is the method of the 107^{th} embodiment, wherein the audio sound track is characterized by a track rhythm and by a track beat.

An 109^{th} embodiment is the method of one of the 107^{th} and the 108^{th} embodiments, wherein the audio sound track is familiar to the subject.

An 110^{th} embodiment is the method of one of the 108^{th} and the 109^{th} embodiments, wherein the audio sound track comprises a song, a music album, an audio book chapter, an audio book, a recited poem, a collection of recited poems, or combinations thereof.

An 111^{th} embodiment is the method of one of the 108^{th} through the 110^{th} embodiments, wherein the method further comprises receiving additional sensor data from one or more wearable sensors configured to be worn by a subject, wherein the additional sensor data comprise motion data, sound data, physiological data, or combinations thereof of the subject; determining that the additional sensor data deviates from an ordinary profile associated with the subject; responsive to the determination that the sensor data deviates from the ordinary profile associated with the subject, delivering the audible sound therapy to the subject, wherein the audible sound therapy comprises the audio sound track associated with the subject.

An 112^{th} embodiment is a method comprising (a) acquiring sensor data from one or more wearable sensors configured to be worn by a subject, wherein the sensor data comprise motion data, sound data, physiological data, or combinations thereof of the subject; (b) comparing the sensor data of the subject with target data, wherein the motion data, the sound data, the physiological data, or combinations thereof of the subject are compared with target motion data, target sound data, target physiological data, or combinations thereof, respectively; (c) determining, in any sequence, at least one of the following: that the motion data of the subject is equal to or exceed the target motion data; that the sound data of the subject is equal to or exceed the target sound data; and that the physiological data of the subject is equal to or exceed the target physiological data; and (d) responsive to step (c), delivering audible sound therapy to the subject, wherein the audible sound therapy comprises an audio sound track, wherein the audio sound track is characterized by a track rhythm and by a track beat, wherein the audio sound track is familiar to the subject, and wherein the audio sound track is repeated at least until it is determined, in any sequence, at least one of the following: that the target motion data exceed the motion data of the subject; that the target sound data exceed the sound data of the subject; and that the target physiological data exceed the physiological data of the subject.

An 113^{th} embodiment is the method of the 112^{th} embodiment, wherein the subject has a neurodevelopmental disorder comprising autism spectrum disorder (ASD), sensory processing disorder, or combinations thereof; wherein, when the subject has ASD, the sensor data being equal to or exceeding target data correlates with the onset of a pre-meltdown stage for the subject.

An 114^{th} embodiment is the method of the 113^{th} embodiment, wherein the step of delivering audible sound therapy to the subject prevents the onset of a meltdown stage for the subject or decreases the severity of a meltdown stage for the subject.

An 115^{th} embodiment is the method of one of the 111^{th} through the 114^{th} embodiments, wherein the audio sound track comprises a song, a music album, an audio book chapter, an audio book, a recited poem, a collection of recited poems, or combinations thereof.

An 116^{th} embodiment is the method of one of the 111^{th} through the 115^{th} embodiments, wherein the motion data comprise motion frequency and/or motion intensity; wherein the target motion data comprise target motion frequency and/or target motion intensity, respectively; and wherein, when the motion data of the subject is equal to or exceed the target motion data, the audible sound therapy is delivered to the subject at least until the target motion frequency and/or target motion intensity exceed the motion frequency and/or motion intensity, respectively, of the subject.

An 117^{th} embodiment is the method of one of the 111^{th} through the 116^{th} embodiments, wherein the motion data comprise motion frequency and/or motion intensity; wherein the target motion data comprise target motion frequency and/or target motion intensity, respectively; and wherein, when the motion data of the subject is equal to or exceed the target motion data, the audible sound therapy is delivered to the subject at least until the target motion frequency and/or target motion intensity exceed the motion frequency and/or motion intensity, respectively, of the subject.

An 118^{th} embodiment is the method of one of the 111^{th} through the 117^{th} embodiments, wherein the sound data comprise vocal sounds produced by the subject; and wherein, when the sound data of the subject is equal to or exceed the target sound data, the audible sound therapy is delivered to the subject at least until the target sound data exceed the sound data of the subject.

An 119^{th} embodiment is the method of one of the 111^{th} through the 118^{th} embodiments further comprising receiving, by a control system, the sensor data from the one or more wearable sensors; wherein the control system comprises at least one processor and at least one controller; wherein the at least one processor compares the sensor data to the target data; wherein, when at least one of the sensor data are equal to or exceed the target data, the at least one processor signals the at least one controller; and wherein the at least one controller delivers the audible sound therapy to the subject.

An 120^{th} embodiment is the method of the 119^{th} embodiment, wherein the control system provides for real-time delivery of the audible sound therapy to the subject.

An 121^{st} embodiment is the method of one of the 119^{th} and 120^{th} embodiments, wherein the control system selects the audio sound track that is familiar to the subject from a library of audio sound tracks that are familiar to the subject; and wherein said selection is based on the type of sensor data of the subject that is equal to or exceed the target data and/or the magnitude of difference between the sensor data of the subject and the target data.

An 122^{nd} embodiment is the method of the 121^{st} embodiment, wherein acquiring sensor data from one or more wearable sensors further comprises receiving, by at least one computing device, the sensor data from the one or more wearable sensors; wherein the at least one computing device comprises the at least one processor; wherein comparing the sensor data of the subject with target data further comprises providing, by the at least one computing device, an input to a model that is trained with sensor data from the subject and/or with sensor data from at least one additional subject; wherein the model is configured to evaluate the sensor data of the subject with respect to the target data to determine the presence or the absence of a pre-meltdown stage for the subject; wherein the at least one processor, based upon a determination of the presence of a pre-meltdown stage for the subject, signals the at least one controller; and wherein the at least one controller delivers the audible sound therapy to the subject.

An 123^{rd} embodiment is the method of the 122^{nd} embodiment, wherein evaluating the sensor data of the subject with respect to the target data further comprises determining, by the at least one computing device, that the sensor data of the subject is equal to or exceed the target data; wherein determining, by the at least one computing device, that the sensor data of the subject is equal to or exceed the target data further comprises comparing at least one evaluation score with at least one threshold score value and determining that the evaluation score is equal to or exceeds the threshold score value.

An 124^{th} embodiment is the method of the 123^{rd} embodiment, wherein step (d) of delivering audible sound therapy to the subject further comprises delivering audible sound therapy to the subject by the at least one controller at least until the threshold score value exceeds the evaluation score.

An 125^{th} embodiment is a system comprising one or more wearable sensors configured to detect sensor data of a subject; wherein the one or more wearable sensors comprise at least one sensor configured to detect motion data, at least one sensor configured to detect sound data, at least one sensor configured to detect physiological data, or combinations thereof; and a control system configured to receive the sensor data of the subject from the one or more wearable sensors; wherein the control system comprises at least one processor and at least one controller; wherein the at least one processor compares the sensor data to target data; wherein, when at least one of the sensor data are equal to or exceed the target data, the at least one processor is configured to signal the at least one controller; and wherein the at least one controller delivers an audible sound therapy to the subject; wherein the audible sound therapy comprises an audio sound track, wherein the audio sound track is characterized by a track rhythm and by a track beat, wherein the audio sound track is familiar to the subject, and wherein the audio sound track is repeated at least until it is determined that the target data exceed the sensor data of the subject.

An 126^{th} embodiment is the system of the 125^{th} embodiment, wherein the at least one processor compares the motion data, the sound data, the physiological data, or combinations thereof of the subject with target motion data, target sound data, target physiological data, or combinations thereof, respectively.

An 127^{th} embodiment is the system of one of the 125^{th} and the 126^{th} embodiments, wherein the control system further comprises (i) at least one machine learning model that is trained with sensor data from the subject and/or with data from at least one additional subject, wherein the at least one machine learning model is configured to evaluate the sensor data of the subject with respect to target data; and (ii) a non-transitory computer readable medium that stores instructions that when executed by the processor, causes the processor to: receive, using the control system, an input comprising sensor data of the subject; provide, by the control system, the input to the at least one machine learning model; determine, by the control system, an evaluation result comprising an indication that the sensor data of the subject is equal to or exceed the target data by using the at least one machine learning model; wherein the indication that the sensor data of the subject is equal to or exceed the target data is an evaluation score being equal to or greater than a threshold score value; and deliver, by the control system, audible sound therapy to the subject, wherein the audible sound therapy comprises an audio sound track, wherein the audio sound track is familiar to the subject, and wherein the audio sound track is configured to be repeated at least until the target data exceed the sensor data of the subject.

An 128^{th} embodiment is the system of one of the 125^{th} through the 127^{th} embodiments, further comprising an audio speaker, earbuds, and/or headphones, wherein the audible sound therapy is delivered via the audio speaker, earbuds, and/or headphones without requiring assistance from a caregiver.

An 129^{th} embodiment is the system of one of the 125^{th} through the 128^{th} embodiments, wherein the subject has autism spectrum disorder (ASD); wherein the sensor data being equal to or exceeding target data correlates with the onset of a pre-meltdown stage for the subject; and wherein the audible sound therapy prevents the onset of a meltdown stage for the subject or decreases the severity of a meltdown stage for the subject.

An 130^{th} embodiment is a method comprising delivering audible sound therapy to a subject, wherein the audible sound therapy comprises an audio sound track, wherein the audio sound track comprises a song, a music album, an audio book chapter, an audio book, a recited poem, a collection of recited poems, or combinations thereof, and wherein the audio sound track is characterized by a track rhythm and by a track beat; receiving sensor data from one or more wearable sensors worn by the subject, wherein the sensor data comprise motion data, sound data, physiological data, or combinations thereof of the subject that is indicative of a state of the subject during delivery of the audible sound therapy to the subject; determining that the audio sound track is effective to prevent onset of a meltdown stage for the subject or decrease severity of a meltdown stage for the subject; and associating the audio sound track with the subject.

An 131^{st} embodiment is the method of the 130^{th} embodiment, wherein the method further comprises receiving additional sensor data from one or more wearable sensors configured to be worn by a subject, wherein the additional sensor data comprise motion data, sound data, physiological data, or combinations thereof of the subject; determining that the additional sensor data deviates from an ordinary profile associated with the subject; responsive to the determination that the sensor data deviates from the ordinary profile associated with the subject, delivering the audible sound therapy to the subject, wherein the audible sound therapy comprises the audio sound track associated with the subject.

While embodiments of the disclosure have been shown and described, modifications thereof can be made without departing from the spirit and teachings of the invention. The embodiments and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

What is claimed is:

1. A method comprising:
(a) acquiring sensor data from one or more wearable sensors configured to be worn by a subject, wherein the sensor data comprise motion data, sound data, physiological data, or combinations thereof of the subject;
(b) comparing the sensor data of the subject with target data, wherein the motion data, the sound data, the physiological data, or combinations thereof of the subject are compared with target motion data, target sound data, target physiological data, or combinations thereof, respectively;
(c) determining, in any sequence, at least one of the following: that the motion data of the subject is equal to or exceed the target motion data; that the sound data of the subject is equal to or exceed the target sound data; and that the physiological data of the subject is equal to or exceed the target physiological data; and
(d) responsive to step (c), delivering audible sound therapy to the subject, wherein the audible sound therapy comprises an audio sound track, wherein the audio sound track is characterized by a track rhythm and by a track beat, wherein the audio sound track is familiar to the subject, and wherein the audio sound track is repeated at least until it is determined, in any sequence, at least one of the following: that the target motion data exceed the motion data of the subject; that the target sound data exceed the sound data of the subject; and that the target physiological data exceed the physiological data of the subject.

2. The method of claim 1, wherein the subject has a neurodevelopmental disorder comprising autism spectrum disorder (ASD), sensory processing disorder, or combinations thereof; wherein, when the subject has ASD, the sensor data being equal to or exceeding target data correlates with the onset of a pre-meltdown stage for the subject.

3. The method of claim 2, wherein the step of delivering audible sound therapy to the subject prevents the onset of a meltdown stage for the subject or decreases the severity of a meltdown stage for the subject.

4. The method of claim 1, wherein the audio sound track comprises a song, a music album, an audio book chapter, an audio book, a recited poem, a collection of recited poems, or combinations thereof.

5. The method of claim 1, wherein the motion data comprise motion frequency and/or motion intensity; wherein the target motion data comprise target motion frequency and/or target motion intensity, respectively; and wherein, when the motion data of the subject is equal to or exceed the target motion data, the audible sound therapy is delivered to the subject at least until the target motion frequency and/or target motion intensity exceed the motion frequency and/or motion intensity, respectively, of the subject.

6. The method of claim 1, wherein the physiological data comprise heart rate, blood pressure, respiration rate, breathing pattern, oxygen saturation rate, muscle tension level, temperature, one or more electrocardiogram (ECG) features, one of more electromyogram (EMG) features, or combinations thereof; and wherein, when the physiological data of the subject is equal to or exceed the target physiological data, the audible sound therapy is delivered to the subject at least until the target physiological data exceed the physiological data of the subject.

7. The method of claim 1, wherein the sound data comprise vocal sounds produced by the subject; and wherein, when the sound data of the subject is equal to or exceed the target sound data, the audible sound therapy is delivered to the subject at least until the target sound data exceed the sound data of the subject.

8. The method of claim 1 further comprising receiving, by a control system, the sensor data from the one or more wearable sensors; wherein the control system comprises at least one processor and at least one controller; wherein the at least one processor compares the sensor data to the target data; wherein, when at least one of the sensor data are equal to or exceed the target data, the at least one processor signals the at least one controller; and wherein the at least one controller delivers the audible sound therapy to the subject.

9. The method of claim 8, wherein the control system provides for real-time delivery of the audible sound therapy to the subject.

10. The method of claim 8, wherein the control system selects the audio sound track that is familiar to the subject from a library of audio sound tracks that are familiar to the subject; and wherein said selection is based on the type of sensor data of the subject that is equal to or exceed the target data and/or the magnitude of difference between the sensor data of the subject and the target data.

11. The method of claim 10, wherein acquiring sensor data from one or more wearable sensors further comprises receiving, by at least one computing device, the sensor data from the one or more wearable sensors; wherein the at least one computing device comprises the at least one processor; wherein comparing the sensor data of the subject with target data further comprises providing, by the at least one computing device, an input to a model that is trained with sensor data from the subject and/or with sensor data from at least one additional subject; wherein the model is configured to evaluate the sensor data of the subject with respect to the target data to determine the presence or the absence of a pre-meltdown stage for the subject; wherein the at least one processor, based upon a determination of the presence of a pre-meltdown stage for the subject, signals the at least one controller; and wherein the at least one controller delivers the audible sound therapy to the subject.

12. The method of claim 11, wherein evaluating the sensor data of the subject with respect to the target data further comprises determining, by the at least one computing device, that the sensor data of the subject is equal to or exceed the target data; wherein determining, by the at least one computing device, that the sensor data of the subject is equal to or exceed the target data further comprises comparing at least one evaluation score with at least one threshold score value and determining that the evaluation score is equal to or exceeds the threshold score value.

13. The method of claim 12, wherein step (d) of delivering audible sound therapy to the subject further comprises delivering audible sound therapy to the subject by the at least one controller at least until the threshold score value exceeds the evaluation score.

14. A system comprising:
one or more wearable sensors configured to detect sensor data of a subject; wherein the one or more wearable sensors comprise at least one sensor configured to detect motion data, at least one sensor configured to detect sound data, at least one sensor configured to detect physiological data, or combinations thereof; and
a control system configured to receive the sensor data of the subject from the one or more wearable sensors; wherein the control system comprises at least one processor and at least one controller; wherein the at least one processor compares the sensor data to target data; wherein, when at least one of the sensor data are equal to or exceed the target data, the at least one processor is configured to signal the at least one controller; and wherein the at least one controller delivers an audible sound therapy to the subject; wherein the audible sound therapy comprises an audio sound track, wherein the audio sound track is characterized by a track rhythm and by a track beat, wherein the audio sound track is familiar to the subject, and wherein the audio sound track is repeated at least until it is determined that the target data exceed the sensor data of the subject.

15. The system of claim 14, wherein the at least one processor compares the motion data, the sound data, the physiological data, or combinations thereof of the subject with target motion data, target sound data, target physiological data, or combinations thereof, respectively.

16. The system of claim 14, wherein the control system further comprises (i) at least one machine learning model that is trained with sensor data from the subject and/or with data from at least one additional subject, wherein the at least one machine learning model is configured to evaluate the sensor data of the subject with respect to target data; and (ii) a non-transitory computer readable medium that stores instructions that when executed by the processor, causes the processor to: receive, using the control system, an input comprising sensor data of the subject; provide, by the control system, the input to the at least one machine learning model; determine, by the control system, an evaluation result comprising an indication that the sensor data of the subject is equal to or exceed the target data by using the at least one machine learning model; wherein the indication that the sensor data of the subject is equal to or exceed the target data is an evaluation score being equal to or greater than a threshold score value; and deliver, by the control system, audible sound therapy to the subject, wherein the audible sound therapy comprises an audio sound track, wherein the audio sound track is familiar to the subject, and wherein the audio sound track is configured to be repeated at least until the target data exceed the sensor data of the subject.

17. The system of claim 14 further comprising an audio speaker, earbuds, and/or headphones, wherein the audible sound therapy is delivered via the audio speaker, earbuds, and/or headphones without requiring assistance from a caregiver.

18. The system of claim 14, wherein the subject has autism spectrum disorder (ASD); wherein the sensor data being equal to or exceeding target data correlates with the onset of a pre-meltdown stage for the subject; and wherein the audible sound therapy prevents the onset of a meltdown stage for the subject or decreases the severity of a meltdown stage for the subject.

19. A method comprising:
delivering audible sound therapy to a subject, wherein the audible sound therapy comprises an audio sound track, wherein the audio sound track comprises a song, a music album, an audio book chapter, an audio book, a recited poem, a collection of recited poems, or combinations thereof, and wherein the audio sound track is characterized by a track rhythm and by a track beat;
receiving sensor data from one or more wearable sensors worn by the subject, wherein the sensor data comprise motion data, sound data, physiological data, or combinations thereof of the subject that is indicative of a state of the subject during delivery of the audible sound therapy to the subject;
determining that the audio sound track is effective to prevent onset of a meltdown stage for the subject or decrease severity of a meltdown stage for the subject; and
associating the audio sound track with the subject.

20. The method of claim 19, wherein the method further comprises:
receiving additional sensor data from one or more wearable sensors configured to be worn by a subject, wherein the additional sensor data comprise motion data, sound data, physiological data, or combinations thereof of the subject;
determining that the additional sensor data deviates from an ordinary profile associated with the subject;
responsive to the determination that the sensor data deviates from the ordinary profile associated with the subject, delivering the audible sound therapy to the subject, wherein the audible sound therapy comprises the audio sound track associated with the subject.
